(19) United States
(12) Patent Application Publication
(10) Pub. No.: US 2026/0294753 A1
(43) Pub. Date: Oct. 1, 2026

(54) Nanoemulsion and Moisturizing Cleansing Composition Comprising the Same

(71) Applicant: Conopco, Inc., d/b/a UNILEVER, Hoboken, NJ (US)

(72) Inventors: Congling QUAN, Woodbridge, CT (US); Teanoosh MOADDEL, Watertown, CT (US)

(73) Assignee: Conopco, Inc., d/b/a UNILEVER, Hoboken, NJ (US)

(21) Appl. No.: 19/093,675

(22) Filed: Mar. 28, 2025

(51) Int. Cl. A61K 8/06; A61K 8/44; A61K 8/46; A61Q 5/02; A61Q 19/00; A61Q 19/10

(52) U.S. Cl. CPC A61K 8/068; A61K 8/44; A61K 8/466; A61Q 5/02; A61Q 19/007; A61Q 19/10; A61K 2800/21; A61K 2800/30; A61K 2800/596

(57) ABSTRACT

The invention is directed to a mild cleansing or wash composition that delivers excellent moisturizing sensory attributes. The cleansing composition comprises oil droplets provided in a nanoemulsion. The cleansing composition lathers well and can be formulated with mild surfactants and substantially free of sulfate-based surfactants.

### FIELD OF THE INVENTION

The present invention is directed to a nanoemulsion and a moisturizing cleansing composition that delivers highly desirable sensory attributes and that comprises the nanoemulsion. More particularly, the cleansing composition comprises an anionic surfactant mixture that includes an acyl isethionate. The cleansing composition also comprises amphoteric surfactant whereby the cleansing composition comprises no more than 12.3% by weight total surfactant. The cleansing composition in addition to being moisturizing, surprisingly, lathers and rinses well, even when formulated with as much as 8.5% by weight total occlusive. Such a cleansing composition does not leave skin feeling tacky or draggy, is stable and may be formulated substantially free of at least one of sulfate-based surfactants, dioxanes, parabens, hydantoins, phthalates, soaps, acrylate-derived thickeners, dyes, silicones and isothiazolinones.

### BACKGROUND OF THE INVENTION

Wash compositions are typically employed to cleanse skin and to reduce shine associated with sebum produced in epithelial cells known as sebocytes. They are also used to minimize bacteria on the hands and face such that washing is viewed as the most effective way to prevent the spread of germs and bacteria. In fact, experts believe that periodic washing throughout the day can reduce the number of consumers catching colds by about 50%.

Since the Covid-19 pandemic, consumers are more wash conscious, and therefore, attracted to wash compositions that not only help reduce the chances of getting sick, but also minimize a dry and stripped skin feel. Negative feedback associated with moisturizing wash compositions suggest that typical compositions lather poorly, and therefore, are associated with a less impactful cleansing experience. Additional feedback suggests such conventional moisturizing compositions (i.e., those having occlusive droplets in the micron size range) can leave the skin feeling tacky and are often hard to evacuate from packaging in view of the characteristics of the occlusive used.

It is of increasing interest to develop a moisturizing cleansing composition that lathers well, and that results in consumer desirable sensory attributes without leaving skin feeling tacky or draggy. It is also of increasing interest to develop a product that is easier to evacuate from packaging and that is less reliant on high levels of occlusives with droplets in the micron size range, and especially, occlusives derived from petroleum.

The present invention, therefore, is directed to a nanoemulsion and a moisturizing cleansing composition that comprises the nanaoemulsion whereby the cleansing composition surprisingly delivers highly desirable consumer moisturizing sensory attributes, the cleansing composition having an anionic surfactant mixture comprising an acyl isethionate. Such a composition also comprises amphoteric surfactant whereby the cleansing composition comprises no more than 12.3% by weight total surfactant. The cleansing composition in addition to being moisturizing, surprisingly, lathers and rinses well, even when formulated with as much as 8.5% by weight total occlusive. Such a cleansing composition does not leave skin feeling tacky or draggy, is stable and may be formulated substantially free of at least one of sulfate-based surfactants, dioxanes, parabens, hydantoins, phthalates, soaps, acrylate-derived thickeners, dyes, silicones and isothiazolinones.

Additional Information Efforts have been disclosed for making cleansing compositions. In U.S. Pat. No. 9,066,859, viscoelastic cleansing gels having nonionic and amphoteric surfactants are described.

Other efforts have been disclosed for making cleansing compositions. In U.S. Pat. No. 9,974,726, gentle cleansing compositions with make-up removal properties are described.

Even other efforts have been disclosed for making cleansing compositions. In World Application Nos. WO 2020/023187A1 and WO 2020/099036A1, personal cleansing compositions are described.

Still other efforts have been disclosed for making cleansing compositions. In U.S. Pat. No. 10,039,939 and U.S. Published Patent Application No. 2018/0318195 A1, sulfate-free cleansing compositions are described.

None of the additional information describes a nanoemulsion and moisturizing cleansing composition as described and claimed herein.

### SUMMARY OF THE INVENTION

In a first aspect, the present invention is directed to an oil-in-water nanoemulsion comprising:
a) from 1.5 to 8.5%, and preferably, from 2 to 8.5%, and most preferably, from 2.5 to 8.5% or from 3 to 8% or from 3.5 to 8% or from 4 to 8% or from 4 to 7.5% or from 4.5 to 7.5% by weight anionic surfactant, the anionic surfactant comprising 100% by weight acyl isethionate or 100% by weight acyl taurate based on total weight of the anionic surfactant, or acyl isethionate, acyl taurate or both and co-anionic surfactant, the co-anionic surfactant making up from 0.01 to 65%, and preferably, from 5 to 60%, and most preferably, from 20 to 58% or from 25 to 55% or from 30 to 55% or from 40 to 55% or from 45 to 55% or from 47 to 54% or from 0.1 to 11% or from 6 to 11% by weight of the total weight of anionic surfactant in the nanoemulsion, the co-anionic surfactant not being an acyl isethionate or acyl taurate;
b) from 0.0 to 5.5%, and preferably, from 0.1 to 5.5%, and most preferably, 1 to 5%, or from 1.2 to 4.75%, or from 1.5 to 3.5%, or from 1.5 to 3.25%, or from 1.5 to 3%, or from 1.65 to 2.5%, or from 1.75 to 2.25% by weight amphoteric surfactant;
c) from 0.2 to 5.5%, and preferably, from 0.5 to 3.5%, and most preferably, from 1 to 3.5% or from 1 to 3% or from 1 to 2.5% or from 1 to 2% or from 1.2 to 1.75% or from 1.2 to 1.65% of a C₈-C₁₈, and preferably, C₁₀-C₁₆, and most preferably, C₁₂-C₁₄ fatty acid, fatty alcohol, fatty amide, or a mixture thereof;
d) from 40 to 70%, and preferably, from 45 to 68%, and most preferably, from 50 to 65%, or from 55 to 65%, or from 57 to 64% by weight non-hydrogenated oil, or from 40 to 70%, and preferably, or from 45 to 68%, and most preferably, or from 50 to 65%, or from 55 to 65%, or from 57 to 64% by weight of a mixture of non-hydrogenated oil and hydrogenated oil, the mixture comprising no more than 37%, or from 0.0001 to 36%, or from 5 to 36%, or from 10 to 35%, or from 15 to 34% by weight hydrogenated oil based on total weight of the mixture of non-hydrogenated and hydrogenated oil;
e) from 13 to 55%, and preferably, from 17 to 48%, and most preferably, from 20 to 45%, or from 20 to 40%, or from 22 to 35%, or from 24 to 33% by weight water, with the provisos that the nanoemulsion:
i) has a pH from 5.3 to 7.5, and preferably, from 5.8 to 7, and most preferably, from 6 to 6.9, or from 6.1 to 6.8;
ii) is substantially free of at least one of sulfate-based surfactants, phthalates, soaps, dioxanes, parabens, hydantoins and isothiazolinones;
iii) has total surfactant that does not exceed 11.5%, and preferably, comprises from 4 to 10.5%, and most preferably, from 4.3 to 10%, or from 4.5 to 8% by weight surfactant;
iv) has the oil at a droplet size from 10 to 775 nm, and preferably, from 20 to 550 nm, and most preferably, or from 45 to 350 nm, or from 45 to 300 nm, or from 50 to 250 nm, or from 60 to 275 nm, or from 75 to 255 nm, or from 80 to 180 nm; and
v) comprises from 42 to 75%, or from 50 to 70%, or from 50 to 68%, or from 55 to 68%, or from 55 to 65% by weight oil.

In a second aspect, the present invention is directed to a lamellar cleansing composition comprising:
a) anionic surfactant comprising from 1.8 to 6.75%, and preferably, from 2 to 5.5%, and most preferably, from 2 to 5%, or from 2. to 4.75%, or from 2.2 to 4.5%, or from 2.25 to 4.25%, or from 2.25 to 4%, or from 2.25 to 3.5% by weight of acyl isethionate, and from 1.8 to 6.75%, and preferably, from 2 to 5.5%, and most preferably, from 2 to 5%, or from 2.25 to 4.75%, or from 2.25 to 4.5%, or from 2.25 to 4.25%, or from 2.3 to 4%, or from 2.3 to 3.5%, or from 2.3 to 3% by weight of a co-anionic surfactant;
b) from 3 to 7.5%, and preferably, from 3.5 to 7%, and most preferably, from 3.75 to 6.75%, or from 4 to 6.5%, or from 4.25 to 6.25%, or from 4.5 to 6.2%, or from 4.65 to 6.2% by weight amphoteric surfactant;
c) less than 1.25%, and preferably, less than 1%, and most preferably, less than 0.8%, or from 0.0 to 0.75%, or from 0.1 to 0.5%, or from 0.2 to 0.35% by weight electrolyte;
d) from 0.3 to 6.5%, and preferably, from 1 to 5.75%, and most preferably, from 2.5 to 5.5%, or from 2.75 to 5.2%, or from 2.75 to 5%, or from 3.5 to 5%, or from 3 to 4.75% by weight thickener based on total weight of the cleansing composition;
e) 0 to 4.5%, or from 0.001 to 4%, or from 0.01 to 3.5%, or from 0.1 to 3.3%, or from 1 to 3% by weight micro-oil; and
f) nano-oil
   with the provisos that:
   i) at least 40%, or from 45 to 100%, or from 50 to 100%, or from 60 to 100%, or from 70 to 100%, or from 80 to 100%, or from 40 to 92%, or from 45 to 90%, or from 55 to 88%, or from 65 to 88%, or from 75 to 88%, or from 50 to 65%, or from 52 to 63% or 100% by weight of total oil based on total weight of micro-oil and nano-oil in the cleansing composition is provided from nanoemulsion according to the first aspect of the invention;
   ii) the total weight of surfactant in the cleansing composition is from 6.5% to 12.3%, or from 7 to 12.3%, or from 8.5% to 12.3%, or from 9 to 12%, or from 9.2 to 11.8%, or from 9.4 to 11.5%, or from 9.5 to 11%, or from 9.5 to 10.8% by weight of the cleansing composition;
   iii) the cleansing composition is substantially free of sulfate-based surfactant;
   iv) at least 50%, or from 60 to 100%, or from 70 to 100%, or from 70 to 98%, or from 75 to 95%, or from 85 to 95%, or 100% by weight of hydrophobic chain present on the amphoteric surfactant is triglyceride derived based on total weight of the hydrophobic chain on the amphoteric surfactant;
   v) the cleansing composition has a pH from 5.0 to 7.5, and preferably, from 5.6 to 7, and most preferably, from 6.0 to 6.9, or from 6.1 to 6.8; and
   vi) the cleansing composition comprises from 0.5 to 5.5%, and preferably, from 1 to 5%, and most preferably, from 1 to 4.5% or from 1 to 4% or from 1 to 3.5% or from 1 to 3.2% or from 1.2 to 3% or from 1.6 to 2.85% of a C₈-C₁₈, and preferably, C₁₀-C₁₈, and most preferably, C₁₂-C₁₈ fatty acid, fatty alcohol, fatty amide, or a mixture thereof.

In a third aspect, the present invention is directed to a method for cleansing a surface by contacting the surface with the lamellar cleansing composition of the second aspect of the invention, applying shear to generate lather and wiping the surface, or rinsing the surface with water or both.

In a fourth aspect, the present invention is directed to the use of nanoemulsion, anionic and amphoteric surfactants in a lamellar wash composition to impart moisturizing sensory attributes to skin.

All other aspects of the present invention will more readily become apparent from the description and examples which follow.

Skin, as used herein, is meant to include skin on the arms (including underarms), face, back, feet, neck, chest, hands, legs, buttocks and scalp. Surface is meant to include skin, nails and hair. Stable means no visible syneresis, no gelation, no precipitation and able flow as a homogeneous composition even after being stored at 22° C. for at least one month (preferably, one year) and 50° C. for at least two weeks, and as visually determined by skilled and trained panelists.

Oil, as used herein, means an occlusive suitable for use in a composition that may be topically applied to a surface, including skin. Such oil may be saturated (hydrogenated) or unsaturated (non-hydrogenated) or a mixture saturated and unsaturated oils. The oil used may be naturally derived, synthetically manufactured or a mixture thereof. In an embodiment of the invention, no more than 40%, and preferably, no more than 30%, and most preferably, no more than 20% of the oil used in the nanoemulsion and cleansing composition of the present invention is petroleum derived. In yet another embodiment, from 0.001 to 15% by weight of the total weight of oil used in the nanoemulsion and cleansing composition is derived from petroleum. In still another embodiment, all (100% by weight) of the oil used in the present invention is sustainable and plant derived. Micro-oil means having a droplet size from 1 to 500 microns, and preferably, from 1 to 425 microns, and most preferably, from 1 to 400 microns in size, or from 2 to 375 microns, or 3 to 370 microns, or from 4 to 360 microns, or from 5 to 355 microns, or from 6 to 350 microns, or from 10 to 300 microns, or from 10 to 250 microns, or from 11 to 150 microns, or from 11 to 85 microns, or from 1 to 40 microns, or from 1 to 35 microns or from 1 to 28 microns or from 2 to 25 microns in size or from 1 to 11 microns. Nano-oil means oil having a droplet size under 1 micron. Fully hydrogenated oil means having an iodine value from 0 to 20, and preferably, 0 to 15, and most preferably, 0 to 10, or from 0 to 5, or from 0 to 2 with a hydrocarbon chain length from 12 to 38, or from 14 to 38, or from 16 to 38 or from 12 to 36.

Oil used without a qualifier means or includes oil for use in the nanaoemulsion, and oil of micron droplet size suitable for use in the cleansing composition with nanoemulsion (i.e., micro-oil and nano-oil). Droplet size, (volume moment mean diameter D[4,3]) may be obtained following the conventions described in ASTM E 799, Practice for Determining Data Criteria and Processing for Drop Size Analysis. For the avoidance of doubt, D[4,3] is the De Brouckere mean, which is taken by dividing the sum of the 4^{th} power of Di* percentage by the 3^{rd} power of the same calculation. Regarding the oil suitable for use, in an embodiment of the invention, such oil can be selected from hydrogenated and nonhydrogenated triglyceride, hydrogenated and nonhydrogenated mono-ester oil, hydrogenated and nonhydrogenated naturally occurring oil other than triglyceride and including shea butter, and partially hydrogenated triglyceride oil. In another embodiment of the invention, less than 45%, and preferably, less than 42%, and most preferably, from 15 to 42%, or from 18 to 40% by weight of the total oil used in the nanoemulsion and wash composition has a fully saturated (100% sp³ hybridized) hydrophobic portion.

Surfactants described herein will typically have an HLB from 7.7 to 20, or from 8 to 18, or from 10 to 18, or from 10 to 18, or from 10 to 17, where HLB is the hydrophilic-lipophilic balance of a surfactant and is calculated via the formula HLB=20*Mₕ/M, Mₕ being the molecular mass of the hydrophilic portion of the molecule and M is the molecular mass of the whole molecule.

Palm kernel oil derived and coconut oil derived mean that a component has a fatty portion (i.e., hydrophobic chain), for example, with C₈-C₁₆ saturated hydrocarbon chains obtained from palm kernel oil or coconut oil (as the case may be). Occlusive means an additive or ingredient for a composition and used to enhance the trapping or securing of moisture to or within (or both) a surface like skin and is synonymous with oil. Lamellar, as used herein, means having layers of surfactant in arrangement where polar head groups align with water to shield fatty acid acyl chains from the water such that over 85% (or 90 to 100% or 100%) of the total resulting surfactants in the composition are arranged as micelle plates or layers creating a translucent or opaque composition. In the present invention and as is demonstrated in the data provided, it has been unexpectedly discovered that compositions consistent with the invention, in addition to being stable, surprisingly result in superior lathering characteristics when washing as determined by trained panelists comparing the cleansing (or wash) composition of the present invention to control compositions having similar surfactant amounts and with 0.0 to no more than 2% by weight oil or occlusive. Washing is performed with the wash compositions being compared and with composition to water at a weight ratio of 1.4:1 to 1:1.4 or from 1.2:1 to 1:1.2 or from 1:1 where water is typically used at a temperature from 35 to 40° C., or from 36 to 39° C., or from 36 to 38° C.

The cleansing composition of the present invention is one which can have a viscosity from 500 mPa-s to 2,000,000 mPa-s where viscosity is taken at 25° C. with a Discovery HR-2 Rheometer using a parallel plate or cone and plate having a 1000-micron gap and a shear rate of 0.1 s-1, unless stated otherwise.

The cleansing composition is one suitable to be wiped or washed off, and preferably, washed off with water. Such a composition can be a home care cleaning composition (e.g., for tabletops, glass, toilets dishes, upholstery, or laundry) but is preferably a shampoo, make-up wash, facial wash, or hand wash, and especially, a personal care liquid body wash. In an embodiment of the invention, the cleansing composition can have a viscosity from 1,500 to 1,800,000 mPa-s, and preferably, from 5,000 to 1,675,000 mPa-s, and most preferably, from 8,000 to 1,450,000 mPas, or from 9,000 to 1,175,000 mPa-s, or from 10,000 to 1,115,000 mPa-s, or from 15,000 to 1,000,000 mPa-s, or from 17,000 to 1,000,000 mPa-s, or 17,500 to 950,000 mPa-s, or from 3,000 to 16,000 mPa-s, or from 3,500 to 13,000 mPa-s. When used for hand applications, the lamellar cleansing composition of the present invention will preferably have a viscosity that is not higher than 15,000 mPa-s, preferably not over 10,000 mPa-s, and most preferably, between 2,000 and 8,500 mPa-s.

The cleansing composition of this invention may, optionally, comprise medicinal or therapeutic agents, but preferably, is a wash which is for cosmetic uses, non-therapeutic and formulated to remove dirt, oil or the like from surfaces including skin and hair. In a preferred embodiment, the lamellar cleansing composition is a personal wash composition, and therefore, a liquid body wash for use while showering or bathing. The lamellar cleansing composition of the present invention may optionally comprise skin benefit ingredients added thereto such as vitamins and/or derivatives thereof, resorcinols, retinoic acid precursors, activator of peroxisome proliferator-activated receptors (PPARs), colorants, moisturizers including humectants, sunscreens, antibacterial agents, mixtures thereof or the like. Such skin benefit ingredients (or agents) can be water and/or oil soluble. If used, oil soluble skin benefit agents typically make up to 2% (or preferably up to 1.5%, or from 0.001 to 1%, or from 0.01 to 0.3%) by weight of the lamellar cleansing composition whereby water-soluble skin benefit agents, when optionally used, may make up to 15% (or up to 10%, or up to 5%, or from 0.01 to 4.5%) by weight of the cleansing composition. The cleansing composition will typically have a pH from 5.0 to 7.5, and preferably, from 6 to 7, and most preferably, from 6.1 to 6.9, or from 6.1 to 6.8.

In an embodiment of the invention, the nanoemulsion described herein may be added as an ingredient at an amount of 1 to 11%, and preferably, from 1.3 to 10%, and most preferably, from 1.3 to 9.5%, or from 1.4 to 9.2%, or from 1.8 to 9%, or from 2 to 8.5% by weight of the total weight of the cleansing composition. Composition used generally herein is meant to mean the nanoemulsion and lamellar cleansing composition. As to the nanoemulsion, when added as an ingredient or component to make the cleansing composition, nano-oil will homogeneously disperse in the resulting cleansing composition, resulting in superior skin moisturization upon use.

As used herein, “substantially free of” as it relates to sulfates (i.e., sulfate-based surfactants like sodium lauryl sulfate and ethoxylated surfactants like sodium lauryl ether sulfate) means less than 3.0%, and preferably, less than 2%, and most preferably, less than 1%, even more preferably, less than 0.75% or less than 0.5% or less than 0.2% by weight of the cleansing composition. In an embodiment of the invention, substantially free of sulfate includes a wash composition with 0.0% (none) by weight sulfate-based surfactant (—OSO₃⁻ comprising). Regarding the non-sulfate-based ingredients that the cleansing compositions can be substantially free of (e.g., ingredients, like DMDM hydantoin preservative), substantially free of means less than 0.8% by weight, and preferably, less than 0.7%, and most preferably, less than 0.5%, or less than 0.3% or less than 0.22% or less than 0.1% or less than 0.05% by weight of the cleansing composition. As to the non-sulfate-based ingredients, substantially free of also includes 0.0% (i.e., none) by weight of the ingredient in the cleansing composition. With respect to all ingredients that the cleansing composition of the present invention may be substantially free of, it is within the scope of the invention to include from 0.001 to 0.065% or from 0.01 to 0.045% by weight of such ingredients. Substantially free of as used herein is, for the avoidance of doubt, meant to mean as applied to each ingredient individually. In yet another embodiment, the cleansing composition comprises less than 35 ppm, and preferably, less than 25 ppm, and most preferably, less than 15 ppm dioxane or less than 2 ppm or less than 1 ppm dioxane or less than 0.05 ppm or 0.0 ppm dioxane based on total dioxane in the cleansing composition where dioxane includes 1,4-dioxane. In another embodiment, the cleansing composition of this invention may comprise from 0.00001 to 0.00005% by weight dioxane, like 1,4-dioxane.

Amphoteric surfactant means a surfactant that changes from positive or cationic to negative or anionic as the pH goes from acidic to basic wherein the term amphoteric as used herein is meant to include zwitterionic surfactants, surfactants that can simultaneously maintain both a cationic and anionic charge at a certain pH.

Both the nanoemulsion and wash composition of the present invention comprise less than 3%, or less than 2%, or less than 1.5%, of less than 1%, or from 0.0001 to 0.5%, or less than 0.4%, or 0.0% by weight surfactant having an HLB under 7.7, or under 7.5, or from 4 to 7.6 where HLB is as previously defined.

Unless explicitly stated otherwise, all ranges described herein are meant to include all ranges subsumed therein. The term comprises is meant to encompass the terms consisting essentially of and consisting of. For the avoidance of doubt and for illustration, a composition comprising an isethionate and betaine is meant to include, for example, a composition consisting essentially of the same and a composition consisting of the same. As to the percentages used herein, the same are meant to be by weight of ingredient (i.e., not including any carrier, like water, that may be supplied with the ingredient) unless noted otherwise. Except in the operating and comparative examples, or where otherwise explicitly indicated, all numbers used in this description indicating amounts, or ratios of materials and/or use thereof are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

As to the isethionates that may be used in the nanoemulsion and cleansing composition, such anionic surfactants include C₆-C₂₀ acyl isethionates. These surfactants (esters) are prepared by a reaction between alkali metal isethionate with mixed aliphatic fatty acids having from 6 to 20 carbon atoms and an iodine value of less than 20, or less than 10. Often at least 75% of the mixed fatty acids have from 12 to 18 carbon atoms and up to 25% can have 6 to 10 carbon atoms. The acyl isethionate suitable for use may be an alkoxylated isethionate such as those described in Ilardi et al., U.S. Pat. No. 5,393,466, entitled “Fatty Acid Esters of Polyalkoxylated isethonic acid. The isethionate surfactants can include the reaction product of fatty acids esterified with isethionic acid and neutralized with a base like sodium or potassium hydroxide. The acyl isethionate surfactant can satisfy the general formula:

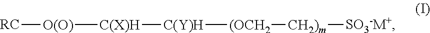

where R is an alkyl group having 5 to 19 carbons, m is an integer from 0 to 4 (1 to 4 for the alkoxylated option), X and Y are each independently hydrogen or an alkyl group having 1 to 4 carbons and M is hydrogen, ammonium, alkali metal cation, a lower C₁ to C₄, alkanol ammonium cation and/or a basic amino acid cation. In an embodiment of the invention, M includes sodium, ammonium and/or potassium ions.

It is also within the scope of the invention, when m is 0, for either the carbon alpha or beta to the sulfonate group to have one C₁₋₄ alkyl substitution in place of hydrogen, preferably a C₁₋₃ alkyl substitution, and most preferably, a methyl group. It is also within the scope of the invention for R to have a degree of unsaturation and typically no more than 2, and more preferably, no more than 1 double bond. Furthermore, and optionally, it is within the scope of the invention for the alpha or beta carbon, or both present on the R group to be substituted or branched with a C₁₋₃ alkyl group, with the proviso that both of such alpha and beta carbons are not simultaneously disubstituted. In the embodiment of the invention, the branching group is a methyl group.

Illustrative examples of the isethionates suitable for use include sodium capryl isethionate, sodium caproylyl isethionate, sodium capryl methyl isethionate, sodium caproylyl methyl isethionate, sodium cocoyl isethionate, sodium cocoyl methyl isethionate, sodium lauroyl isethionate, sodium lauroyl methyl isethionate, potassium lauroyl isethionate, potassium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, ammonium cocoyl isethionate, ammonium cocoyl methyl isethionate, or mixtures thereof. In an embodiment of the invention, the isethionate used in the present invention is sodium lauroyl isethionate.

Taurate suitable for use in the nanoemulsion and lamellar cleansing composition include, for example, those which are acylamides of taurine or N-methyltaurine, and salts thereof. For example, taurates suitable for use are acyl taurates represented by the general formulae:

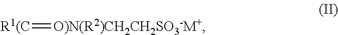

where R¹ is C₅ to C₂₉, more particularly, C₅ to C₂₃ alkyl, R² is hydrogen or methyl, and M is as previously defined. In one embodiment, R¹ is C₅ to C₁₇ alkyl. In another embodiment at least half of the R¹ groups are C₇-C₁₈ alkyl. In still another embodiment at least half of the R¹ groups are C₉ to C₁₅ alkyl, or C₉ to C₁₃ alkyl. R¹ may be saturated or unsaturated, or branched in the manner described for the isethionates depicted by structure I. In yet another embodiment R² is methyl. For the avoidance of doubt, overall alkyl chain lengths described herein will include the carbonyl carbon.

Illustrative taurates that may be used as anionic surfactant include, for example, taurates commonly known as sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, mixtures thereof or the like. In an embodiment of the invention and when used, the taurate selected is sodium methyl lauroyl taurate, sodium methyl cocoyl taurate or a mixture thereof. In another embodiment, the taurate used is sodium methyl lauroyl taurate.

In still another embodiment of the invention, at least 55%, and preferably, at least 75%, and most preferably, at least 80% or 90 to 100%, or 92 to 99%, or 94 to 97% or 95 to 99% or 100% by weight of the total anionic surfactant used in the nanoemulsion is acyl isethionate, acyl taurate or a mixture of acyl isethionate and acyl taurate. In another embodiment, the anionic surfactant in the nanoemulsion is all acyl isethionate or a mixture of acyl isethionate and acyl taurate where acyl taurate makes up from 18 to 35%, or from 20 to 32%, or from 22 to 30%, or from 22 to 28% by weight of the mixture of acyl isethionate and acyl taurate.

Additional anionic surfactants suitable to use in the nanoemulsion and cleansing composition of the present invention include those classified as acyl glutamates and acyl glycinates.

Acyl glutamates (and salts thereof) suitable for use include C₈ to C₂₀, and preferably, C₁₀ to C₁₈ and most preferably, C₁₂ to C₁₆ or C₁₂ to C₁₄ acyl glutamates where the acyl portion is preferably saturated but suitable to be unsaturated with no more than 2 double bonds, including conjugated double bonds. Illustrative yet nonlimiting examples of the glutamates that may be used include sodium capryloyl glutamate, sodium lauroyl glutamate, sodium myristoyl glutamate, sodium cocoyl glutamate, sodium stearoyl glutamate, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium olivoyl glutamate, sodium palmitoyl glutamate, sodium undecylenoyl glutamate, disodium cocoyl glutamate or a mixture thereof. In an embodiment of the invention, a mixture of at least two of sodium lauroyl glutamate, sodium cocoyl glutamate and sodium stearoyl glutamate is preferred. In another embodiment, if a glutamate is used, sodium myristoyl and sodium cocoyl glutamate are often desired, either individually or as a mixture.

As to the acyl glycinates (and salts thereof) suitable for use, these include C₈ to C₂₀, and preferably, C₁₀ to C₁₈ and most preferably, C₁₂ to C₁₆ or C₁₂ to C₁₄ acyl glycinates. Illustrative and nonlimiting examples of the glycinates that may be used include sodium lauroyl glycinate, sodium myristoyl glycinate, sodium cocoyl glycinate, potassium lauroyl glycinate, sodium myristoyl glycinate, potassium cocoyl glycinate or a mixture thereof. In an embodiment of the invention, sodium cocoyl glycinate, potassium cocoyl glycinate or a mixture thereof are often preferred when acyl glycinates are used. In another embodiment, sodium or potassium lauroyl glycinate or both may also be used in the lamellar wash compositions of the invention. As with the glutamates suitable for optional use, the acyl portion of the glycinates optionally used is preferably saturated but suitable to be unsaturated with no more than 2 double bonds, including conjugated double bonds.

Other anionic surfactants suitable for use include alkyl sulfates, alkyl ether sulfates, alkyl sulfonates, alpha-olefin sulfonates, alkyl sulfosuccinates, alkyl ether sulfosuccinates, acyl sarcosinates or a mixture thereof (including any salts thereof).

As noted herein, the lamellar wash composition of the present invention is preferably substantially free of sulfate-based surfactants. In an embodiment of the invention sulfate-based surfactant may be used at an amount of less than 3% by weight of the total weight of the lamellar cleansing composition. If optionally included, the sulfate-based surfactants can include C₈-C₂₀ alkyl sulfates and/or C₈-C₂₀ alkyl ether sulfates. When selected, sodium lauryl sulfate, sodium lauryl ether sulfate, ammonium lauryl sulfate, ammonium lauryl ether sulfate, sodium pareth sulfate or a mixture thereof are often selected for use. As to those sulfate-based surfactants classified as alkyl ether sulfates, the ethoxy portion is typically from 1 to 3 ethoxy units in length, and often, from 2 to 3 ethoxy units in length.

As to the anionic sulfonates and their salts that may be selected for use (in nanoemulsion and cleansing composition), the same can include alkyl sulfonates, alkyl glyceryl ether sulfonates, alkyl alpha olefin sulfonates (hydrocarbons being an alkene, CₓH₂ₓ, with a double bond in the alpha position) or a mixture thereof. Typically, the alkyl portion is a C₈-C₂₄ hydrocarbon, and preferably, from C₁₀-C₂₀ hydrocarbon, and more preferably, from C₁₂ to C₁₈ or from C₁₂ to C₁₆ or from C₁₄ to C₁₆ hydrocarbon.

Suitable succinates (including their salts) that may be included are those with a C₁₀ to C₂₀ hydrophobic portion. Illustrative examples include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a mixture thereof. MIPA and MEA refer to monoisopropanolamine and monoethanolamine, respectively.

The acyl sarcosinates suitable for use include those having a C₈-C₂₀ or C₁₀-C₁₈ or C₁₂-C₁₈ acyl group. Illustrative examples of the sarcosinates that may be used include sodium lauroyl sarcosinate, sodium cocoyl sarcosinate, or a mixture thereof.

In an embodiment of the invention, at least one anionic surfactant used is selected from sodium lauryl sulfosuccinate, sodium myristoyl sulfosuccinate, sodium cocoyl sulfosuccinate, sodium stearoyl sulfosuccinate, sodium laureth sulfosuccinate, sodium pareth sulfosuccinate, disodium laureth sulfosuccinate, disodium lauryl sulfosuccinate, diethylhexyl sodium sulfosuccinate or a mixture thereof.

In yet another embodiment, the anionic surfactant used can include sodium methyl 2-sulfolaurate or disodium 2-sulfolaurate or both. Still another anionic surfactant that may be used is an alaninate like sodium cocoyl alaninate, sodium myristol alaninate, sodium cocoyl methyl alaninate, sodium lauroyl aspartate, sodium myristoyl aspartate, sodium cocoyl aspartate or a mixture thereof.

For the avoidance of doubt, mixtures of any of the anionic surfactants may be used and the cation portion of their salts can include sodium, potassium and ammonium ions or mixtures thereof.

In still another embodiment of the invention, often-desired anionic surfactants suitable for use are those generally classified as lactylates, including C₁₀-C₂₀ lactylates. Such lactylates can be a mono- or polylactyl or a mixture thereof as lactic acid can, for example, undergo self-esterification. Therefore, C₁₀-C₂₀ lactylates suitable for use include lactylic esters of fatty acids represented by the formula:

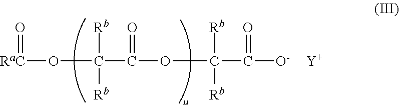

where R^{a} is a C₉ to C₁₉ hydrocarbon, each Rbis independently hydrogen or a C₁₋₃ alkyl with at least one R^{b} being alkyl (preferably methyl), u is an integer from 0 to 3 and Y⁺ is a counter ion that can include K⁺, Na⁺, NH₄⁺ or a mixture thereof.

In an embodiment of the invention, the C₁₀-C₂₀ lactylate comprises 40 to 100%, and preferably, 50 to 95%, and most preferably, 60 to 90% (or 65 to 85% or 70 to 80%) by weight of a C₁₂-C₂₀ (or C₁₄-C₂₀ or C₁₆-C₂₀ or C₁₆-C₁₈) group (i.e., acyl portion) based on total weight of lactylate if optionally used in the lamellar wash compositions. The preferred lactylates, when used, are C₁₄-C₂₀ lactylates, and more preferably, C₁₆-C₁₈ lactylates like palmitoyl-1-, stearoyl-1-lactylate or mixtures thereof. Polylactyls (typically numbering from two to three lactyl groups) are also suitable for use, like palmitoyl-2-lactylate, stearoyl-2-lactylate or mixtures thereof. In an embodiment of the invention, sodium lauroyl lactylate, sodium stearoyl lactylate or mixtures thereof are the preferred optional lactylates.

Yet another anionic suitable for optional use is when the R^{b} groups are hydrogen and the anionic represented by formula (III) is a glycolate.

As noted, the nanoemulsion comprises from 1.5 to 8.5%, and preferably, from 2 to 8.5%, and most preferably, from 2.5 to 8.5% or from 3 to 8% or from 3.5 to 8% or from 4 to 8% or from 4 to 7.5% or from 4.5 to 7.5% by weight anionic surfactant, the anionic surfactant comprising 100% by weight acyl isethionate or 100% by weight acyl taurate based on total weight of the anionic surfactant, or acyl isethionate, acyl taurate or both and co-anionic surfactant, the co-anionic surfactant making up from 0.01 to 65%, and preferably, from 5 to 60%, and most preferably, from 20 to 58% or from 25 to 55% or from 30 to 55% or from 40 to 55% or from 45 to 55% or from 47 to 54% or from 0.1 to 11% or from 6 to 11% by weight of the total weight of anionic surfactant in the nanoemulsion, the co-anionic surfactant not being an acyl isethionate or acyl taurate.

In an embodiment of the invention, the anionic surfactant included in the nanoemulsion will comprise from 80 to 100% or from 85 to 100% or from 85 to 95% or 95 to 100% or 100% by weight sodium lauroyl isethionate and sodium methyl lauroyl taurate where sodium methyl lauroyl taurate makes up from 20 to 35% or from 22 to 32% or from 22.5 to 30% by weight of the total weight of anionic surfactant in the nanoemulsion.

As to the cleansing composition comprising the nanoemulsion of the present invention, such cleansing composition will comprise anionic surfactant comprising from 1.8 to 6.75%, and preferably, from 2 to 5.5%, and most preferably, from 2 to 5% or from 2. to 4.75% or from 2.2 to 4.5% or from 2.25 to 4.25% or from 2.25 to 4% or from 2.25 to 3.5% by weight of acyl isethionate, and from 1.8 to 6.75%, and preferably, from 2 to 5.5%, and most preferably, from 2 to 5% or from 2.25 to 4.75% or from 2.25 to 4.5% or from 2.25 to 4.25% or from 2.3 to 4% or from 2.3 to 3.5% or from 2.3 to 3% by weight of a co-anionic surfactant based on total weight of the wash composition where any of the anionic surfactants other than an acyl isethionate may be used as co-anionic surfactant. Co-anionic surfactant, therefore, means an anionic surfactant other than an acyl isethionate.

In an embodiment of the invention, the anionic surfactant in the wash composition will comprise sodium lauroyl isethionate, and at least one of a taurate, glycinate, alaninate, sarcosinate, glutamate or a mixture thereof. In another embodiment, the anionic surfactant will comprise sodium lauroyl isethionate and sodium lauroyl glycinate at a weight ratio of 1.5:1 to 1:1.5, or from 1.3:1 to 1:1.3 or from 1.1:1 to 1:1.1 or 1:1 where the total weight of sodium lauroyl isethionate and sodium lauroyl glycinate is from 3 to 6.5% or from 3.5 to 6% or from 4 to 5.5% by weight of the cleansing composition.

The amphoteric surfactants suitable for use in the present invention comprise C₆ to C₂₀, and preferably, from C₆ to C₁₈, and most preferably, from C₈ to C₁₈ hydrophobic chains (i.e., making up the hydrophobic portion) where from 2.5 to 35%, and preferably, from 3 to 32%, and most preferably, from 3.5% to 30%, or from 3.8 to 28%, or from 4 to 20%, or from 5 to 19%, or from 6 to 18%, or from 8 to 15% of the total weight of amphoteric surfactant is C₈ to C₁₀ hydrophobic chain and the C₈ chain to C₁₀ chain amphoteric surfactants are present at a weight ratio from 1:12 to 12:1 or from 1:7.5 to 7.5:1 or from 1:5 to 5:1 or from 1:2.5 to 2.5:1 or from 1:1.5 to 1.5:1 or from 1:1.25 to 1.25:1 or from 1:1.15 to 1.15 to 1 or 1:1. Such amphoteric surfactants will also comprise from 38 to 55%, and preferably, from 40 to 53%, and most preferably, from 42 to 52%, or from 42 to 48%, or from 42 to 46% by weight C₁₂ hydrophobic chain based on total weight of the amphoteric surfactant used.

As to the specific amphoteric surfactants noted herein, the same will have a mixture of hydrophobic chains as defined with surfactant having the C₁₂ hydrophobic chain making up from 38 to 55% by weight of the total weight of amphoteric surfactant in the nanoemulsion and lamellar cleansing composition. For the avoidance of doubt and for illustration, when the amphoteric surfactant selected is cocamidopropyl betaine, the betaine will have C₁₂ hydrophobic chain making up from 38 to 55% by weight of the total weight of betaine in the lamellar cleansing composition, subject to the additional chain distributions described.

The amphoteric surfactants suitable for inclusion in the compositions of the present invention (i.e., the nanoemulsion and lamellar cleansing composition), subject to the noted chain distributions, include those with at least one acid group. The acid group may be a carboxylic or a sulphonic acid group. They often include quaternary nitrogen, and therefore, can be quaternary amino acids. Such surfactants should generally include an alkyl or alkenyl group of 6 to 18 carbon atoms and generally comply with the overall structural formula:

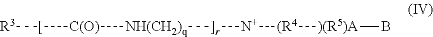

where R³ is alkyl or alkenyl of 5 to 19 carbon atoms; R⁴ and R⁵ are each independently alkyl, hydroxyalkyl or carboxyalkyl of 1 to 3 carbon atoms; q is 2 to 4; r is 0 or 1; A is an alkylene of 1 to 3 carbon atoms optionally substituted with hydroxyl, whereby B is —CO₂— or —SO₃—.

Suitable amphoteric surfactants that may be used in the present invention and within the above general formula include simple betaines of formula:

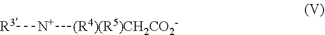

and amido betaines of formula:

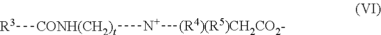

where t is 2 or 3.

In both formulae R³, R⁴ and R⁵ are as previously defined. R³ would, in particular, include a mixture of C₇ to C₁₇ alkyl groups and R⁴ and R⁵ are preferably methyl or ethyl groups, most preferably methyl groups. R^{3′} is C₅ to C₁₇.

A further possibility is that the amphoteric surfactant is a sulphobetaine of the formula:

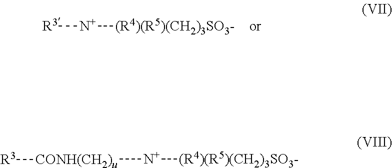

where u is 2 or 3, or variants of these in which —(CH₂)₃SO₃⁻ is replaced by —CH₂C(OH)(H)CH₂SO₃⁻.

In these formulae (VII and VIII), R³, R^{3′}, R⁴ and R⁵ are as previously defined.

Illustrative examples of the amphoteric surfactants suitable for use include betaines like lauryl betaine, laurylhydroxy sulfobetaine, lauryldimethyl betaine, coco betaine, cocoamidopropylhydroxylsulfo betaine, cocodimethyl carboxymethyl betaine, cocamidopropyl betaine, laurylamidopropyl betaine, cocodimethyl carboxymethyl betaine, mixtures thereof or the like.

Additional amphoteric surfactants suitable for use include lauryl hydroxysultaine, cocamidopropyl hydroxy sultaine or mixtures thereof. Such surfactants are made commercially available, and it is within the scope of the invention to employ mixtures of the aforementioned surfactants. In a preferred embodiment, the amphoteric surfactant used in the wash of this invention is triglyceride derived cocamidopropyl betaine, and again, subject to the noted chain distributions.

In an embodiment of the invention, less than 20%, and preferably, less than 35%, and most preferably, less than 50% or less than 70% or less than 85% or less than 95% by weight of the hydrophobic portion of the amphoteric surfactants are recovered from petroleum, palm oil, palm kernel oil and/or coconut oil. In another preferred embodiment, 100% of the hydrophobic portion of the amphoteric surfactants is not recovered from petroleum, palm oil, palm kernel oil, coconut oil or a mixture thereof.

In even another embodiment of the invention, from 0.0 to 15% or from 0.05 to 12% or from 0.5 to 10% by weight of any of the surfactants used in the compositions may have hydrophobic portion with carbon recovered from purple carbon, and that is, carbon recovered from carbon dioxide waste gas via biotechnology that utilizes microbial gas fermentation.

In yet another embodiment, at least 10% or at least 25%, and preferably, at least 40%, and most preferably, from 40 to 100%, or from 50 to 100% or from 55 to 95% or 100% by weight of the amphoteric surfactants used can have hydrophobic portion recovered from triglycerides such as those recovered from jojoba, avocado, olive, and nuts, as well as from seed oil (e.g., sunflower, linseed, rapeseed), and especially, from soy bean oil.

As to the nanoemulsion, amphoteric surfactant, when used, will typically make up from 1.2 to 5.5%, and preferably, from 1.4 to 5%, and most preferably, from 1.5 to 4.75% or from 1.6 to 4.5% or from 1.65 to 4.25%, or from 1.7 to 3.5% or from 1.75 to 3% or from 1.8 to 2.5% by weight of the nanoemulsion. In an embodiment of the invention, the nanoemulsion can comprise less than 0.5% by weight amphoteric surfactant or no amphoteric surfactant, 0.0% by weight of the nanoemulsion. Regarding the cleansing composition, amphoteric surfactant will typically make up from 1.8 to 8.5%, and preferably, from 2 to 8%, and most preferably, from 2.5 to 7.75% or from 3 to 7% or from 3.5 to 6.5%, or from 4 to 6.2% or from 4.2 to 6% or from 4.5 to 5.6% by weight of the cleansing composition.

In an embodiment of the invention, at least 75%, and preferably, at least 85%, and most preferably, at least 90% (or 90 to 100%, or 92 to 99%, or 94 to 97% or 95 to 99% or 100%) by weight of the total weight of amphoteric surfactant used in the compositions is a surfactant having the chain distribution as described herein.

Optional amphoteric surfactants may be used and these, if desired, will make up less than 25% by weight of the total weight of amphoteric surfactant in the compositions. Preferably, such optional amphoteric surfactants make up less than 20%, or less than 15% or from 0.001 to 12% or from 0.01 to 9% or from 0.01 to 5% by weight of the total weight of amphoteric surfactant in the compositions. In an embodiment of the invention, only amphoteric surfactant (i.e., 100% by weight of the amphoteric surfactant) satisfying the herein defined chain distribution is used in the compositions of this invention. Optional amphoteric surfactant means surfactant that is typically fatty acid derived and not derived from triglyceride.

Optional amphoteric surfactants that may be included and that do not contain hydrophobic portion that is triglyceride derived include, for example, cocamidopropyl betaine that has fatty acid derived or obtained hydrophobic portion. If included, other suitable examples are C₁₆₋₂₀ amidopropyl hydroxysultaines where the C₁₆₋₂₀ amidopropyl hydroxysultaine is preferably palmityl, stearyl and/or oleyl amidopropyl hydroxysultaine, and most preferably, palmityl amidopropyl hydroxysultaine.

Even other suitable suitable surfactants that may optionally be used include behenyl betaine, capryl/capramidopropyl betaine, stearyl betaine, oleyl betaine, myristyl hydroxysultaine or a mixture thereof.

Even other optional amphoteric surfactants that can be used at levels from 0.01 to less than 25% of the total amphoteric used are cocoyl amine oxide, lauramine oxide, myristamine oxide, palmitamine oxide, stearamine oxide, oleamine oxide, cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof.

Additional amphoteric surfactants suitable for optional use include imidazolines, sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion. Illustrative examples of the amphoteric surfactants suitable for use include sodium lauroamphoacetate, sodium cocoamphoacetate, sodium lauroamphoacetate, sodium cocoamphoacetate, cocamphodipropionate or a mixture thereof.

In still another embodiment of the invention, optional amphoteric selected can include at least 5%, and often, at least 10%, and most preferably, at least 15% (or 15 to 20%) by weight amine oxide based on total weight of amphoteric surfactant used in the compositions whereby such amine oxide can have the formula: R^{x}—N⁺(R^{y})₂—O— where R^{x} is a C₈₋₂₀ alkyl, and preferably, a C₁₀₋₁₈ alkyl, and most preferably, a C₁₂₋₁₈ alkyl (or C₁₂₋₁₆ alkyl) and R^{y} is H or a C₁₋₆ alkyl or C₁₋₄ alkyl or —C₁₋₃ alkyl or C₁₋₂ alkyl or —CH₃. In another embodiment of the invention, amine oxide selected for optional use is cocoyl amine oxide, lauramine oxide, myristamine oxide, palmitamine oxide, stearamine oxide, oleamine oxide or a mixture thereof.

In even another embodiment of the invention, the optional amphoteric surfactant, if used, will make up from 0.0001 to 1.2% or from 0.001 to 1% or from 0.005 to 0.8% by weight of the compositions.

Nonionic surfactants may optionally be used in the compositions of the present invention. When used, nonionic surfactants are typically used at levels as low as 0.001, 0.02 0.03, 0.05, 1, 1.5 or 2% by weight and at levels as high as 2.5 or 3% by weight of the compositions. The nonionics which may be used include the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include dialkyl sulphoxides, or the like.

In an embodiment of the invention, nonionic surfactants selected can include fatty alcohol ethoxylates like Steareth-2, Steareth-10, Ceteth-20, Oleth-20 Beheneth-20 or a mixture thereof. In another embodiment, fatty acid ethoxylates may be used and they include lauric acid ethoxylate, coconut fatty acid ethoxylate, myristic acid ethoxylate, palmitic acid ethoxylate, stearic acid ethoxylate, oleic acid ethoxylate, soya bean fatty acid ethoxylate or a mixture thereof.

Other nonionic surfactants optionally suitable for use may include a sugar amide, such as a polysaccharide amide. Specifically, the surfactant may be one of the lactobionamides as described in U.S. Pat. No. 5,389,279 to Au et al., entitled “Compositions Comprising Nonionic Glycolipid Surfactants issued Feb. 14, 1995. Nonionic surfactant may also include one of the sugar amides described in U.S. Pat. No. 5,009,814 to Kelkenberg, entitled “Use of N-Poly Hydroxyalkyl Fatty Acid Amides as Thickening Agents for Liquid Aqueous Surfactant Systems” issued Apr. 23, 1991.

In an embodiment of the invention, the nonionic surfactant used is an alkyl polyglucoside represented as:
R⁶—O—(S)ₚ, where R⁶ is a C₆ to C₂₀ or C₈ to C₁₈ or C₁₀ to C₁₆ linear or branched alkyl and S is a saccharide group of 5 or 6 carbons such as glucose, xylose, lactose, fructose and/or mannose, and preferably, is glucose, and p represents the degree of polymerization and may have a value of from 1 to 12 or from 1 to 10, and most preferably, from 1.2 to about 1.6 or from 1.3 to 1.5.

In still another embodiment of the invention, the nonionic surfactant used is cocamide monoethanolamine (cocamide MEA), glyceryl monostearate and/or polyglycerol esters like polyglyceryl-8 caprylate, polyglycerol-8 caprate, polyglyceryl-8 myristate, polyglyceryl-8 palmitate, polyglyceryl-9 caprylate, polyglycerol-9 caprate, polyglyceryl-9 laurate, polyglyceryl-9 myristate, polyglyceryl-9 palmitate or a mixture thereof. If used, such nonionics may make up from 0.01 to 3% or from 0.02 to 1.85% or from 0.03 to 1.5% (or from 0.03 to 1%) by weight of the compositions. In another embodiment, the nonionic used is glyceryl monostearate, cocamide MEA, or a mixture thereof at levels from 0.01 to 0.65%, or from 0.01 to 0.5%, or from 0.01 to 0.2% or from 0.01 to 0.07% by weight of the compositions. In still another embodiment, the compositions of the present invention are free of nonionic surfactant, i.e., 0.0% by weight nonionic surfactant.

In still yet another embodiment of the invention, cationic surfactants may optionally be used in the compositions.

One class of optional cationic surfactants that may be used includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants suitable for optional use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl)dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, and strearyl amidopropyl dimethylamine lactate.

Even other useful cationic surfactants suitable for optional use include quaternized hydrolysates of silk, wheat, and keratin proteins, and it is within the scope of the invention to use mixtures of such surfactants.

If used, cationic surfactants will typically make up no more than 1.0% by weight of the compositions. When present, they may make up from 0.001 to 0.7%, and more typically, from 0.01 to 0.5%, or from 0.1 to 0.3% by weight of the compositions. In still another embodiment of the invention, the nanoemulsion and the lamellar cleansing composition of the present invention are free of cationic surfactant, i.e., 0.0% by weight cationic surfactant.

In an embodiment of the invention, thickeners are included in the lamellar cleansing composition of the invention. Thickener used comprises less than 1.5% and preferably, less than 1.25%, and most preferably, less than 1% (or less than 0.5% or from 0.001 to 0.9% or 0.0%) by weight quaternized hydroxyethyl cellulose polymer having a molecular weight from 195 to 825 kDa (or a molecular weight from 185 to 950, or from 170 to 900, or from 150 to 1000 kDa) based on total weight of thickener in the cleansing composition. Such thickener classified as a quarternized hydroxyethyl cellulose like, for example, polyquaternium-67 (SoftCAT™ SK MH) will make up less than 1.5% by weight of total weight of thickener in the lamellar cleansing composition. In another embodiment of the invention, no polyquaternary-67 (i.e., 0.0% by weight) is used in the lamellar cleansing composition of the present invention.

Thickeners often are desired for use in the lamellar cleansing compositions of the present invention. These include polymeric carbohydrate thickeners like nonmodified starch granules, in general, like potato starch, waxy maize starch as well as simple corn starch, i.e., a starch that gelatinizes at around 75° C. Pure-Gel® starches from Grain Processing Corporation are chemically modified corn starch granules having a gelatinization temperature at about 53° C. and are often considered for use. Modified and/or nonmodified starch granules with gelatinization temperatures from 30° to 85° C., and preferably, from 30° to 80° C., and most preferably, from 35 to 75° C. are suitable for inclusion as the polymeric carbohydrate thickening agent in the lamellar cleansing compositions of the invention. In general, and notwithstanding the starch selected for use, it is typically preferred that the granules of the polymeric carbohydrate selected, upon use in the lamellar cleansing composition, swell at least 200%, and preferably at least 400%, and more preferably, at least 600%, and most preferably, at least 800% by volume in the lamellar cleansing composition to form swollen starch gel particles with a size in the range of 2 to 300 micrometers, or preferably, from 3 to 275 microns, and most preferably, from 4 to 245 microns. Examples of thickeners suitable to select are Pure Gel B990, Pure Gel B992, Pure Gel B980 and Pure Dent starches made commercially available from Grain Processing Corporation. Additional polymeric carbohydrates suitable for use (i.e., starch granules) are National™ 1545, Amioca corn starch, Clearjel, National 1333, Colflo 67, Novation 1600, Novation 2700 or Purity 420 made available from Ingredion. Chemically modified starch granules are also suitable for use. Starch granules modified with nonionic hydrophilic groups such as hydroxyethyl or hydroxypropyl and/or ionic groups such as phosphate, carboxylate, sulfate or sulfonate and dialkyl/trialkyl amino groups can also be suitable for use.

Even other polymeric carbohydrates suitable for use are water soluble starches like Ultra-Sperse®, tapioca and waxy maize starch, and National 1215 pregelatinized unmodified corn starch or mixtures thereof. Still others include Structure® ZEA and Structure® (2143 or 6892), hydroxypropyl modified corn starch, or Structure® XL, a cross-linked pregelatinized hydroxypropyl starch phosphate or mixtures thereof, whereby the same are also available commercially from Nouryon. Others like BASF and Cargill provide Cosmedia® HP Starch and StarDesign™ Care, respectively. Such hydroxypropyl starch phosphate has a molecular weight of 238 (C₂₇H₄₈O₁₀), Cas No. 53124-00-8. In an embodiment of the invention, hydroxypropyl starch phosphate is preferred for use.

Other useful thickeners that can be used include carbohydrate gums such as cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum and mixtures thereof; modified and nonmodified starch granules often with gelatinization temperatures between 30 to 85° C.

Additional thickeners that also may be selected for use include Versathix™ (PEG-150 Pentaerythrityl Tetrastearate (and) PPG-2 Hydroxyethyl cocamide and water), Aristoflex AVC (ammonium acryloyldimethyltaurate/VP copolymer), Carbomer such as Carbopol 980 (crosslinked polyacrylic acid), Carbopol® Ultrez 10 and 20 (hydrophobically modified crosslinked polyacrylic acid); alkaline soluble emulsion polymers such as Aculyn 28, Acuyln 22 or Carbopol Aqua SF1; cationic polymer such as modified polysaccharides including cationic guar available from Solvay Novecare under the trade name Jaguar® C13S, Jaguar® C14S, Jaguar® C17, or Jaguar® C16; cationic modified cellulose such as UCARE™ Polymer JR 30M from Dow; N-Hance™ 3000, N-Hance™ 3196, N-Hance™ GPX 215 or N-Hance™ GPX 196 from Ashland, Inc.

In an embodiment, the thickener used comprises no (0.0% by weight of the cleansing composition) cationic thickener like cationic guar polymer. In another embodiment, the lamellar cleansing composition comprises from 0.001 to 0.5% or from 0.001 to 0.4% or from 0.005 to 0.35% or from 0.08 to 0.015% by weight of a thickener that is a cationic like guar polymer. In another embodiment a cationic guar polymer, i.e., guar hydroxypropyltrimonium chloride, (including those with a charge density from 0.8 to 8, and preferably, from 1 to 7.5 or from 1.2 to 7.2 where molecular weight (Mn) ranges from 8,000 to 10,000 million, or from 9,000 to 7 million or from 9,500 to 5.5 million) is used in combination with a starch (preferably a sodium hydroxypropyl starch phosphate) where the total amount of cationic guar polymer used is from 0.1 to 1% or from 0.1 to 0.7% or from 0.1 to 0.5% or from 0.12 to 0.22% by weight of the total weight of the cationic guar polymer and starch used in the compositions. A preferred guar is guar hydroxypropyltrimonium chloride like Jaguar® C14S (guar gum 2-hydroxy-3-trimethylamino propyl ether chloride, CAS No. 65497-29-2).

In yet another embodiment of the invention, less than 1%, and preferably, less than 0.5%, and most preferably, less than 0.25% or less than 0.15% or 0.0% by weight of the thickener used in the cleansing composition is acrylate-based such as those referred to as an acryloyldimethyltaurate/VP copolymer.

The total amount of thickener used in the cleansing composition is often from 0.3 to 6.5%, and preferably, from 1 to 5.75%, and most preferably, from 2.5 to 5.5% or from 2.75 to 5.2% or from 2.75 to 5% or from 3.5 to 5% or from 3 to 4.75% by weight of the lamellar cleansing composition.

In an embodiment of the invention, thickener may optionally be used in the nanoemulsion to aid in adjusting the viscosity of the nanoemulsion where the viscosity of the nanoemulsion is from 1,000 to 600,000 mPa-s, or from 1,500 to 150,000 mPa-s, or from 15,000 to 100,000 mPa-s or from 18,000 to 88,000 mPa-s or from 20,000-70,000 mPa-s.

In yet another embodiment of the invention, the weight ratio of amphoteric surfactant to thickener used in the lamellar cleansing composition is from 1:1.5 to 1.5:1 or from 1:1.3 to 1.3:1 or from 1:1.2 to 1.2:1 or from 1:1.1 to 1.1:1 or from 1:1.

In another embodiment, the cleansing composition of the present invention can comprise an electrolyte such as MgCl₂, CaCl₂), KCl, NaCl or a mixture thereof. In even another embodiment, such electrolyte may be provided alone or with a glycol having the formula:

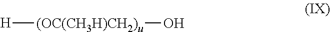

where u is an integer from 6 to 12, and preferably, 7 to 11, or from 8 to 10, and most preferably, 9 (polypropylene glycol 9 (PPG-9)).

Total amount of electrolyte in the cleansing composition is typically less than 1.25%, and preferably, less than 1%, and most preferably, less than 0.8% or from 0.0 to 0.75% or from 0.1 to 0.5% or from 0.1 to 0.5% or from 0.2 to 0.3% by weight electrolyte in the cleansing composition.

Suitable oils (occlusives) for use in the nanoemulsion and lamellar cleansing composition of the present invention include silicone oils and/or mineral oil, but preferably includes oils that are naturally sourced and sustainable like, arachis oil, castor oil, coconut oil, corn oil, cotton seed oil, olive oil, rapeseed oil, safflower seed oil, sesame seed oil, soybean oil, moringa oil, high oleic sunflower oil (at least 80 percent oleic acid, monounsaturated), hydrogenated soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, mixtures thereof or the like. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like.

When dispersing micro-oil in the manufacturing of the lamellar cleansing composition, used is conventional mixing apparatus and moderate to high shear with temperature varying from 28 to 88° C. or from 30 to 85° C., often from 45 to 75° C. Mixing can be continuous or batch as long as the resulting composition is homogeneous, single phase and free of separation and particulate or flocculate sediments. Oil and other wash composition components are combined to form oil droplets (i.e., micro-oil) or particles having a droplet size from 1 to 250 microns, and preferably, from 1 to 100 microns, and most preferably, from 1 to 60 microns in size, or from 1 to 50 microns or 2 to 45 microns, or from 2 to 40 microns, or from 3 to 35 microns, or from 5 to 28 microns, or from 8 to 22 microns, or from 10 to 20 microns, or from 2 to 10 microns, or from 2.5 to 8 microns, or from 1.5 to 12 microns in size.

As to the nanoemulsion, it too may be added or provided in the manufacturing process as or like an ingredient when making the lamellar wash composition where the nanoemulsion is preferably premade before addition and within a water continuous emulsion of submicron droplets within the defined size ranges described herein. As noted such water continuous nanoemulsion typically comprises from 40 to 70%, and preferably, from 45 to 68%, and most preferably, from 50 to 65%, or from 55 to 65% or from 57 to 64% by weight non-hydrogenated oil, or from 40 to 70%, and preferably, from 45 to 68%, and most preferably, from 50 to 65%, or from 55 to 65% or from 57 to 64% by weight of a mixture of non-hydrogenated oil and hydrogenated oil, the mixture comprising no more than 37% or from; 0.0001 to 36%, or from 5 to 36% or from 10 to 35% or from 15 to 34% by weight hydrogenated oil based on total weight of the mixture of non-hydrogenated and hydrogenated oil.

To emulsify the nanoemulsion prior to adding the same as an ingredient in the manufacturing process for the resulting and desired lamellar wash composition, present is typically from 1.5 to 8.5%, and preferably, from 2 to 8.5%, and most preferably, from 2.5 to 8.5% or from 3 to 8% or from 3.5 to 8% or from 4 to 8% or from 4 to 7.5% or from 4.5 to 7.5% by weight anionic surfactant, the anionic surfactant comprising 100% by weight acyl isethionate or 100% by weight acyl taurate based on total weight of the anionic surfactant, or acyl isethionate, acyl taurate or both and co-anionic surfactant, the co-anionic surfactant making up from 0.01 to 65%, and preferably, from 5 to 60%, and most preferably, from 20 to 58% or from 25 to 55% or from 30 to 55% or from 40 to 55% or from 45 to 55% or from 47 to 54% or from 0.1 to 11% or from 6 to 11% by weight of the total weight of anionic surfactant in the nanoemulsion, the co-anionic surfactant not being an acyl isethionate or acyl taurate

Also, present to assist in emulsifying the nanoemulsion is 0.0 to 5.5%, and preferably, from 0.1 to 5.2%, and most preferably, from 1 to 5%, or from 1.2 to 4.75%, or from 1.5 to 3.5%, or from 1.5 to 3.25%, or from 1.5 to 3%, or from 1.65 to 2.5%, or from 1.75 to 2.25% by weight amphoteric surfactant whereby total emulsifier (i.e., surfactant) present within the nanoemulsion does not exceed 11.5% by weight of the nanoemulsion. In an embodiment of the invention, at least one of the surfactants used to emulsify the nanoemulsion is also used as surfactant within the wash composition. In another embodiment, at least two or at least 3 or all of the surfactants or emulsifiers used in the nanoemulsion are identical to the surfactants present in the lamellar cleansing composition.

In still another embodiment, the nanoemulsion comprises less than 1%, and preferably, less than 0.8%, and most preferably, less than 0.5% by weight nonionic surfactant and cationic surfactant. In even another embodiment the nanoemulsion comprises less than 0.2%, and preferably, less than 0.16%, and most preferably, less than 0.14% or from 0.0001 to 0.13% by weight electrolyte based on total weight of the nanoemulsion. In yet another embodiment, the total weight of emulsifier in the nanoemulsion is often from 45 to 78% and preferably, from 48 to 68%, or from 49 to 65%, or from 50 to 62% or from 52 to 60% by weight acyl isethionate, and from 13 to 30%, or from 15 to 27%, or from 17 to 25% by weight each of acyl taurate and alkylamidopropyl betaine.

Suitable oils for use in the nanoemulsion and lamellar cleansing composition of the present invention include silicone oils and/or mineral oil, but preferably includes oils that are naturally sourced and sustainable like, arachis oil, castor oil, coconut oil, corn oil, cotton seed oil, olive oil, rapeseed oil, safflower seed oil, sesame seed oil, soybean oil, hydrogenated soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, mixtures thereof or the like. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like.

Additional oils, like soybean and hydrogenated soybean oil are also suitable for use, as are oils traditionally classified as petroleum jelly, CAS No. 800903-08, whereby such oil is a combination of hydrocarbons mainly having carbon chains longer than 25. Petroleum jelly is characterized as a composition made predominately of the paraffin series that can be obtained by, for example, dewaxing lubricating oil stock (or crude oil refining) whereby the same melts at temperatures from 35 to 72° C. (more often 40 to 70° C.) and boils at a temperature of 285° C. or higher and often at a temperature between 295 and 325° C. Such oil is characterized as a semi-solid at room temperature (22° C.) and spreads well topically at skin's natural temperature of 33 to 37° C. Free of polycyclic aromatics, the most well-known and best produced petroleum jelly is sold under the brand name Vaseline®. As used herein, petroleum jelly and petrolatum are meant to be the same. Semi-solid, as used herein, means soft like Vaseline®, not pourable at room temperature but spreadable on skin at room temperature.

In still another embodiment, the cleansing composition of the present invention comprises less than 0.2% by weight oil or no (0.0% by weight) oil derived from petroleum. In yet another embodiment, less than 0.5% or 0.0% by weight of the total weight of the oil used in the cleansing composition is a silicone oil.

In an embodiment of the present invention, oils selected for use are those which are made to mimic petroleum jelly (“petroleum jelly substitute” or “substitute”) but are not derived from petroleum or any biproduct or residue recovered from the processing of the same such as processing for gas production. They are, therefore, preferably plant-based, sustainable, soft solids that melt at temperatures similar to those described for petroleum jelly (more often from 29 to 76° C.). In an embodiment of the invention, the petroleum jelly substitute used has a melting point from 30 to 62° C. or from 30 to 60° C. or from 30 to 55° C. or from 30 to 50° C. or from 30 to 45° C.

In another embodiment of the invention, at least 50%, or at least 70%, or at least 80% or from 85 to 96% or from 88 to 98% or from 90 to 99% or 95 to 100% or 100% by weight of the micro-oil in the cleansing composition, and nano-oil in the nanoemulsion is soybean and hydrogenated soybean oil. In another embodiment, and especially when total oil used is at least 70% by weight of soybean and hydrogenated soybean oil, the melting point range of the total oil selected is typically from 30-75° C., and preferably, from 35 to 70° C. and most preferably, from 40 to 69° C.

In a further embodiment of the invention, and when the oil used is not 100% by weight soybean and hydrogenated soybean oil, a petroleum jelly substitute is optionally used. Those suitable for use can be a vegetable-based substitute comprising 10 to 60% by weight of a triglyceride; 5 to 40% by weight of castor oil; 10 to 50 percent by weight glycerin; and 0.5 to 10% by weight of polyglyceryl ricinoleate.

In such vegetable-based substitute, triglycerides used may include caprylic/capric triglyceride, coconut oil, sunflower seed oil, safflower oil, cottonseed oil, olive oil or mixtures thereof. Particularly desired is caprylic/capric triglyceride, coconut oil, or both.

Amounts of the triglyceride may range from 10 to about 60%, and preferably, from 20 to 50%, and most preferably, from 30 to 45% by weight of the occlusive (i.e., vegetable-based substitute).

Another component suitable for use in the vegetable-based substitute is castor oil, and particularly, a hydrogenated castor oil, a castor seed oil or combinations thereof. Amounts of castor oil may range from 5 to 40%, and preferably, from 10 to 30%, and most preferably, from 15 to 25% by weight of the vegetable based substitute, and as calculated herein castor oil is not meant to be included in the triglyceride total. In an embodiment of the invention, the nanoemulsion of the present invention comprises from 0.0 to less than 10%, or from 0.0001 to 8%, or from 0.0001 to 7% by weight castor oil.

A further useful component in such substitute is glycerin. Amounts of the same may range from 10 to about 50%, and preferably, from 20 to 40%, and most preferably, from 25 to 35% by weight of the substitute.

An additional component desired in the vegetable-based substitute suitable for use is polyglyceryl ricinoleate. The number of glycerol repeating units may range from 2 to 20, and preferably, from 4 to 10, and most preferably, from 4 to 8 or about 6 repeating units. The term “ricinoleate” is meant to include mono- and poly-ricinoleate esters of glycerol. When the ricinoleate is in poly form, the number of mono ricinoleate units can range from 2 to 20, and preferably, from 2 to 10, or from 2 to 5. Most preferred is polyglyceryl-6 polyricinoleate which has an INCI name of polyglyceryl-6 polyricinoleate. Amounts of this material in the substitute may range from 0.5 to 10%, and preferably, from 1 to 8%, and most preferably, from 4 to 6% by weight of the substitute.

In such substitute it is often desirable to include a fatty acid material like one consisting of trihydroxystearin. Amounts of these materials, when present, may range from 0.1 to 15%, and preferably, from 0.5 to about 10%, and most preferably, from 2 to 8 percent, or from 3 to 6% by weight of the substitute.

Such vegetable-based substitute is substantially free of water, and therefore, comprises from 0 to 5%, and preferably, from 0.001 to 2%, and most preferably, from 0.01 to 1% or 0.0% by weight water based on total weight of the substitute. An additional description of the vegetable-based substitute suitable for use in the present invention may be found in U.S. Pat. No. 8,524,211, the disclosure of which is incorporated herein by reference.

Additional materials that mimic petroleum jelly, i.e., substitutes, suitable for use in the compositions include those generally classified as natural oil substitutes. These oils are the often the esterification product of from 55 to 85% by weight of a fully hydrogenated castor oil and 15 to about 45% by weight of C₈₋₂₂ branched or straight chain fatty acid.

In another embodiment, such substitute includes the esterification product of from 55 to about 85% by weight of fully hydrogenated castor oil, 15 to 45% by weight of C₈₋₂₂ branched or straight chain fatty acid and 5 to 15% by weight of hydrogenated natural oil in addition to castor oil.

Illustrative examples of such natural oils suitable for use in the substitutes include canola oil, avocado seed oil, pumpkin seed oil, rapeseed oil, coconut oil, corn oil, cottonseed oil, olive oil, palm oil, peanut oil, walnut oil, safflower oil, sesame oil, soybean oil, sunflower oil, linseed oil, palm kernel oil, tung oil, jatropha oil, mustard oil, camelina oil, penny cress oil, hemp oil, algal oil, jojoba oil, lard, tallow, poultry fat, yellow grease, fish oil or a mixture thereof.

These substitutes, again, mimic petrolatum or petroleum jelly. The same typically possess a drop melting point measured by AOCS Standard Procedure Cc 18-80 of between 30 to 70° C. and can possess an acid value of less than about 20.0, between 2% to about 7% of combined monoglycerides and diglycerides, an iodine value of less than about 3.0 or any combination of such characteristics. In an embodiment of the invention, such substitute comprises less than 15%, and preferably, less than 10%, and most preferably, less than 5% (or 0.001 to 3.2% or 0.01 to 2.5% or 0.0%) by weight palm kernel oil or coconut oil or both.

In even another embodiment, these natural oil substitutes comprise less than 10% by weight of total monoacylglycerides and diacylglycerides and may comprise from 1 to 8% of a mixture of the same. Such types of natural oil substitutes are described in WO 2309137 A1, the disclosure of which is incorporated herein by reference.

Other substitutes that may be used or included in the present invention include those generally described as:
A natural substitute comprising the esterification product of a pre-esterification mixture that includes 0.1 to 20% by weight of a fatty acid dimer, from 5% to 30% by weight of a C₈₋₂₂ fatty acid, and 65% to 95% of a hydrogenated natural oil, and the petrolatum substitute has an acid value of less than 25.0 as described in WO 22150812 A1, the disclosure of which is incorporated herein by reference;
A natural oil-based petrolatum composition comprising the esterification product of a pre-esterification mixture that comprises 5 to 35% by weight of a fatty acid dimer, 20 to 55% by weight C₈₋₂₂ fatty acid substituted with one or more C₁₋₃ alkyl substituents, about 5 to 20% by weight glycerol, and about 20 to 40% by weight hydrogenated natural oil, wherein the natural based petrolatum product has an acid value of less than 10.0 as described in WO 22150813 A1, the disclosure of which is incorporated herein by reference;
A natural oil-based petrolatum composition comprising the esterification product of a pre-esterification mixture that includes about 15 to 25% by weight fatty acid dimer, based on total weight of the pre-esterification mixture; 15 to 25% by weight glycerin, based on total weight of the pre-esterification mixture; and 55 to 70% by weight of hydrogenated natural oil, based on total weight of the pre-esterification mixture; wherein the natural oil-based petrolatum product has an acid value of less than about 5.0 as described in WO 22150814 A1, the disclosure of which is incorporated herein by reference; and
A natural oil-based petrolatum composition comprising the esterification product of a pre-esterification mixture that includes 0.1 to 40% by weight fatty acid dimer, 60 to 99.9% by weight of one or more components selected from the group consisting of C₂₋₆ polyols, natural oils, hydrogenated natural oils, fatty acids, and acyl glycerols, wherein the natural based petrolatum product has a cone penetration value of greater than 10 and a polydispersity index greater than 1.3 as described in WO 221150815 A1, the disclosure of which is incorporated herein by reference.

In an even another embodiment, the oil used in the present invention can optionally comprise from 5 to 100%, or from 10 to 95% or from 20 to 90% or from 30 to 85% or from 40 to 100% or from 50 to 95% or from 85 to 90% or 100% by weight by shea butter, based on total weight of oil in the nanoemulsion, lamellar cleansing composition or both. Shea butter, for the avoidance of doubt, means triglyceride, predominately oleic and stearic acid recovered via extraction from nuts of the African shea tree, Vitellaria paradoxa.

As to the nanoemulsion with submicron droplets, the same may be made mixing with moderate shear oil, water and emulsifier with heat (typically 50 to 70° C.) to produce a macroemulsion followed by a pass through a high pressure device, such as a high pressure commercially available Sonolator at a pressure of greater than or equal to 1,000 pounds per square inch (6.9 Megapascal (MPa)), and preferably, from 1,500 to 5,000 psi (10.3 to 34.5 MPa), or from 1,500 to 4,500 psi (10.3 to 31 MPa), or from 2,000 to 4,000 psi (13.8 to 27.6 MPa). As to the commercially available Sonolator that may be used to make such emulsion with submicron droplets (i.e., nanoemulsion), they include those made available by Sonic Corporation.

In an embodiment of the invention, a petroleum jelly substitute suitable for use is sold under the name BOTANIJELLY™ and made commercially available by Cargill. Other available oils that may be used as or included as an additive in or substitute for the oils included herein are those sold commercially by Sonneborn under the SonneNatural™ name and including the J-207, NXG, and PF-1 varieties.

In still another embodiment, the oils used in the present invention as petroleum jelly substitutes are preferably used for nano-oil in the nanoemulsion. In yet another embodiment of the invention, such oils can be used as or with other micro-oil within the cleansing composition.

In even another embodiment of the invention, the non-hydrogenated oil includes a liquid typically comprising a triglyceride, monoester oil, hydrocarbon such as squalane, squalene or both, or a mixture thereof. Hydrogenated oil, in addition to hydrogenated triglycerides, can comprise or consist of candelilla wax, carnauba wax, rice bran wax or a mixture thereof.

As to the lamellar wash composition, the same comprises 8.5% by weight total oil or less based on total weight of the wash composition, or from 1.5 to 8%, or from 1.8 to 7%, or from 2 to 6.5%, or from 2.5 to 6%, or from 2.5 to 5%, or from 2.5 to 4.5% by weight oil based on total weight of the wash composition.

The nanoemulsion and lamellar cleansing composition of the present invention can include structuring materials to build viscosity and to enhance composition sensory and the lamellar structure of the cleansing composition. Lamellar, as previously defined does include a two-dimensional phase with lipid bilayers separated by water layers, an opaque, hazy and/or cloudy composition The lamellar cleansing composition of the present invention is one that is creamy, not sticky or draggy, and typically mimics a lotion in consistency. Such a composition also, and surprisingly, maintains excellent fragrance notes for delivery to skin when washing, and provides an enjoyable consumer experience with excellent fragrance notes dissipating when product is deployed from the packing. For the avoidance of doubt and to contrast, isotropic means having lipid layers of one dimension and a transparent composition. Such structuring materials include C₆-C₁₄ acids, C₆-C₁₄ alcohols, C₆-C₁₄ amides (i.e., hydroxylated or amide derivatives of the acids). Often desired for use are caproic, caprylic, capric, lauric or myristic acid or mixtures thereof as well as the alcohol or amide derivatives of the same. In an embodiment of the invention, lauric acid, myristic acid or both are used as structurant or structuring material with lauric acid often being preferred.

When used, the structuring material makes up from 0.5 to 5.5%, and preferably, from 1 to 5%, and most preferably, from 1 to 4.5% or from 1 to 4% or from 1 to 3.5% or from 1 to 3.2% or from 1.2 to 3% or from 1.6 to 2.85% of the lamellar cleansing composition and from 0.4 to 3.3% or from 0.7 to 2.8%, or from 1 to 2.5% or from 1.2 to 2.2% or from 1.2 to 1.9% by weight of the nanoemulsion.

In an embodiment of the invention, the lamellar wash composition comprises less than 4.3%, and preferably, less than 4%, and most preferably less than 3.75%, or from 1.5 to 3.5%, or from 1.6 to 3%, or from 1.8 to 2.85% or from 1.9 to 2.8% by weight of structuring material like caproic, caprylic, capric, lauric or myristic acid or a mixture thereof. In even another embodiment, the lamellar wash composition comprises less than 0.2% by weight, and preferably, less than 0.15, and most preferably, less than 0.1% by weight, or from 0.001 to 0.0015% or from 0.001 to 0.012% by weight guar hydroxypropyltrimonium chloride. In yet another embodiment, the lamellar wash composition of the present invention comprises less than 1.2%, and preferably, less than 1.1%, and most preferably, less than 1% or from 0.2 to 0.9%, or from 0.2 to 0.8%, or from 0.25 to 0.7% by weight electrolyte like NaCl. As herein noted, the amphoteric surfactant preferred for use has at least 50%, or from 60 to 100%, or from 70 to 100%, or from 70 to 98%, or from 75 to 95%, or from 85 to 95%, or 100% by weight of its hydrophobic chain being triglyceride derived based on total weight of the hydrophobic chain on the amphoteric surfactant.

Polyols (i.e., humectants) are suitable for optional use in the lamellar cleansing compositions of the present invention whereby the same are limited only to the extent that they are suitable for use in a topical wash composition. Illustrative and nonlimiting examples of the polyols suitable for use in the present invention include sorbitol, glycerol, mannitol, xylitol, maltitol or mixtures thereof. In an embodiment of the invention, the polyol used is at least 50% by weight glycerol, based on total weight of the polyol used in the wash composition. In another embodiment of the invention, the polyol used is all glycerol (100% by weight). Polyol, when optionally used, will typically make up from 0.25 to 18% by weight of the cleansing composition, and preferably, from 0.3 to 17% by weight of the cleansing composition, and most preferably, from 0.5 to 16% (or from 0.5 to 10%, or from 0.5 to 5% or from 0.5 to 3.75%, or from 0.5 to 2%, or from 0.5 to 1.2% by weight of the lamellar cleansing composition.

Water typically makes up from 55 to 90%, and preferably, from 60 to 85%, and most preferably, from 63 to 82% by weight of the cleansing composition, or from 65 to 82% or from 64 to 80% or from 68 to 80%, or from 72 to 79% by weight of the lamellar cleansing composition. As to the nanoemulsion, water makes up from 13 to 55%, and preferably, from 17 to 48%, and most preferably, from 20 to 45%, or from 20 to 40%, or from 22 to 35%, or from 24 to 33% by weight of the nanoemulsion.

Adjusters suitable to modify/buffer the pH may be used. Such pH adjusters include triethanolamine, NaOH, KOH, H₂SO₄, HCl, C₆ H₈ O₇ (i.e., citric acid) or mixtures thereof. The pH adjusters are added at amounts to yield the desired final pH of the nanoemulsion and cleansing composition as the case may be and as herein defined. The pH values may be assessed with commercial instrumentation such as a pH meter made commercially available from Thermo Scientific®.

As previously described, optional water-soluble skin benefit agents suitable for use in the lamellar cleansing composition of this invention are limited only to the extent that they are capable of being topically applied, and able to dissolve in the wash composition at the desired pH.

Illustrative examples of the skin benefit agents suitable to include in the water portion of the lamellar cleansing composition are acids, like amino acids, such as arginine, valine or histidine. Additional water-soluble benefit agents suitable for use include vitamin B2, niacinamide (vitamin B3), vitamin B5 (Panthenol), vitamin B6, folic acid (vitamin B9), vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, water soluble extracts of sage, aloe vera, green tea, grapeseed, thyme, chamomile, yarrow, cucumber, liquorice, rosemary extract or mixtures thereof. Water soluble sunscreens like ensulizole (phenylbenzimidizole sulfonic acid), disodium phenyl dibenzimidazole tetra sulfonate or mixtures thereof may also be included. Total amount of optional water-soluble benefit agents (including mixtures) when present in the lamellar cleansing composition may range from 0.001 to 15%, preferably from 0.001 to 10%, and most preferably, from 0.01 to 8% (or from 1 to 6% or from 1 to 3%) by weight of the cleansing composition.

It is also within the scope of the present invention to optionally include oil soluble benefit agents (i.e., non-water-soluble agents defined as less than 1.0 g of such benefit agent (solute) can be dissolved in 100 mL of 25° C. water (solvent) at atmospheric pressure). The only limitations with respect to such oil soluble benefit agents are that the same are suitable to provide a benefit when topically applied to a surface, and for example, skin.

Illustrative examples of the types of oil soluble benefit agents that may optionally be used in the cleansing composition of this invention include components like stearic acid, vitamins like Vitamin A, D, E and K (and their oil soluble derivatives), sunscreens like ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-4-hydroxybenzoate or mixtures thereof.

Other well-known sunscreens suitable for use include Mexoryl 400 (methoxypropylamino cyclohexenylidene ethoxyethylcyanoacetate), Mexoryl XL (drometrizole trisiloxane), Mexoryl SX (terephthalylidene dicamphor sulfonic acid), octocrylene (2-ethylhexyl 2-cyano-3,3-diphenylprop-2-enoate) or mixtures thereof.

Other optional oil soluble benefit agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble actives suitable for use include omega-3 fatty acids, omega-6 fatty acids, climbazole, farnesol, ursolic acid, myristic acid, geranyl geraniol, cocoyl hydroxyethyl imidazoline, hexanoyl sphingosine, petroselinic acid, conjugated linoleic acid, terpineol, thymol mixtures thereof or the like.

In an embodiment of the invention, the optional oil soluble benefit agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred. Still another retinoic acid precursor suitable for use is hydroxyanasatil retinoate made commercially available under the name Retextra® as supplied by Molecular Design International. The same may be used in a mixture with the oil soluble actives described herein.

When optional oil soluble agent is used in the composition of the invention, such active typically makes up from 0.001 to 2%, and preferably, from 0.001 to 1.5%, and most preferably, from 0.05 to 1% (or from 0.06 to 0.85%) by weight of the lamellar cleansing composition.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. While traditional preservatives may be used, such as hydantoin derivatives, isothiazolinones, methyl paraben and/or propyl paraben, as described herein the lamellar wash composition of the present invention is preferably substantially free of the same. Other preservatives suitable for use may include iodopropynyl butyl carbamate, phenoxyethanol, hydroxyacetophenone, ethylhexylglycerine, hexylene glycol, imidazolidinyl urea, sodium dehydroacetate, benzyl alcohol, sodium benzoate or mixtures thereof. Even other preservatives suitable for use include sodium dehydroacetate, chlorophenesin and decylene glycol. The preservatives should be selected having regard for the use of the composition and possible incompatibilities between the preservatives and other ingredients in the emulsion. Preservatives are preferably employed in amounts ranging from 0.01% to 2.0% by weight of the total weight of the cleansing composition. Also preferred is a preservative system with hydroxyacetophenone alone or in a mixture with other preservatives. Use of common emollients classified as vicinal diols, like 1,2-octane diol (or other vicinal alkane diols), are also suitable for use with the preservatives described herein and typically at amounts ranging from 0.15 to 1.5%, and preferably, from 0.2 to 1.25%, and most preferably, from 0.25 to 1% (or 0.25 to 0.8%) by weight of the lamellar cleansing composition. In another embodiment of the invention, another preservative suitable for optional use in the present invention comprises capryloyl glycine, undecylenoyl glycine or a mixture thereof.

In an embodiment of the invention, the wash composition is substantially free of hydantoins, parabens, silicones and isothiazolinones as described therein. In still another embodiment, the lamellar cleansing composition of the present invention comprises less than 0.2% by weight silver of a compound, and preferably, less than 0.1% by weight of a silver compound, and most preferably, no silver compounds such as oxides, nitrates, acetates, carbonates and/or citrates of silver, or the like.

In another embodiment, the lamellar cleansing composition of the present invention can optionally comprise at least one of benzethonium chloride, benzalkonium chloride, cetrimonium chloride, zinc pyrithione, chloroxylenol, salicylic acid, citric acid, lactic acid, eugenol, terpineol, thymol, selenium sulfide, piroctone olamine, hinokitiol, linalool, cinnamon oil, lemon grass oil, eucalyptus, vanilla extract, mint extract, zinc pyrithione, orange extract, or a mixture thereof. If any of such are selected for use, the total amount of those selected for use will (in combined total) be from 0.001 to 3.5%, or from 0.01 to 2.5%, or from 0.01 to 1.5%, or from 0.02 to 1% or from 0.03 to 0.8% by weight of the lamellar cleansing composition.

In even another embodiment of the invention, at least one of 1,2-nonanediol tetrahexyldecyl ascorbate, honokiol, hyaluronic acid, climbazole, linalool, anisic acid, levulinic acid, cetylpyridinium chloride, pyruvic acid, hemp seed oil and/or magnolia bark extract is used in the lamellar cleansing composition of the invention. If used, the same either alone or in a mixture makes up from 0.001 to 2%, or from 0.01 to 2%, or from 0.01 to 1.5%, or from 0.02 to 1.4%, or from 0.02 to 1%, or from 0.02 to 0.07% by weight of the lamellar cleansing composition. In even another embodiment, the cleansing composition can optionally be fragrance free and/or comprise less than 0.5% by weight (preferably 0.0% by weight) of dye, phthalate or both.

Fragrances, fixatives, chelators (like EDTA, triethanolamine, sodium phytate, tetra sodium glutamate diacetate and/or sodium gluconate) and exfoliants may optionally be included in the wash composition of the present invention. Each of these substances may range from about 0.03 to about 5%, preferably between 0.01 and 3% (or from 0.02 to 1.2%) by weight of the total weight of the cleansing composition, if used. To the extent the exfoliants are used, those selected should be of small enough particle size so that they do not impede the performance of any packaging used to dispense the compositions of this invention. The exfoliants should also be small enough so that they are gentle on skin. In an embodiment of the invention, the the lamellar cleansing composition is free of (i.e., has 0.0% by weight) chelator.

In even another embodiment of the invention, the cleansing composition of the present invention comprises stearic acid, terpineol, thymol, chloroxylenol or a mixture thereof. In yet another embodiment, the wash composition of the present invention will comprise any combination of colloidal oatmeal, silica (including hydrated silica and rice silica), ascorbic acid and hyaluronic acid. When used, such ingredients typically make up (individually or collectively) from 0.001 to 2.5%, and preferably, from 0.01 to 1.5%, and most preferably, 0.1 to 1% by weight of the lamellar wash composition. In even another embodiment of the invention, the cleansing composition may optionally comprise mandelic acid, caffeine, sodium alicylate or a mixture thereof. If used, such ingredients make up from 0.001 to 1%, or from 0.01 to 0.8%, or from 0.1 to 0.5% by weight of the cleansing composition.

In still another embodiment of the invention, a peroxisome proliferator-activated receptor ligand (“PPAR”) may be included in the cleansing composition. The PPAR used is preferably a lipid PPAR α (alpha) activator such as a C₁₀₋₂₂ saturated fatty acid which is branched or derivatized (i.e., functionalized) with groups like hydroxy groups. The PPAR used can also include a C₁₀₋₂₀ monounsaturated fatty acid and C₁₀₋₂₂ polyunsaturated fatty acids. Corresponding alcohols, triglycerides and phospholipids of any of the noted PPAR acids are also suitable for use in the present invention. These include petroselinic acid, 12-hydroxystearic acid, stearic acid, cis-parinaric acid, trans-7-octadecenoic acid, cis 5,8,11,14,17 eicosapentanoic acid, cis-4,7, 10,13, 16, 19 docosahexenoic acid, columbinic acid, linolenelaidic acid, ricinolaidic acid, stearidonic acid, 2-hydroxystearic acid, 10-hydroxystearic acid, alpha-linolenic acid, arachidonic acid, cis-11,14-eicosadienoic acid, conjugated linoleic acid (c9,t11) or (t10,c12), conjugated linoleic acid (50:50 mix of c9, t11 and t10 c12), coriander acid, linolelaidic acid, monopetroselinic acid, ricinoleic acid, stearolic acid or mixtures thereof.

Additional PPAR alpha activator includes cis-11,14, 17 eicosatrienoic acid, cis-5 eicosenoic acid, cis-8,11,14 eicosatrienoic acid, hexadecatrienoic acid, palmitoleic acid, petroselaidic acid, trans farnesol, cis 13, 16 docosadienoic acid, cis-vaccenic acid, cis-11 eicosenoic acid, cis-13, 16, 19 docosatrienoic acid, cis-13-octadecenoic acid, cis-15-octadecanoic acid, cis-7,10,13,16 docosatetraenoic acid, elaidic acid, gamma-linolenic acid, geranic acid, geranyl geranoic acid, linoleic acid, oleic acid, petroselinyl alcohol, phytanic acid, pinolenic acid, tridecyl salicylic acid or a mixture thereof.

A further suitable category of PPAR alpha activator includes plant extracts, such as biochanin A (red clover phytoestrogen), Chromolaena odorata extract, pomegranate saponifiable hydrolysable extract, buglossoides (stearidonic plant extract), and zanthalene (extract from Sichuan peppercorn). Such PPARs are further described in U.S. Pat. No. 6,423,325, the disclosure of which is incorporated herein by reference.

In a preferred embodiment, the PPAR selected is 12-hydroxystearic acid, stearic acid or a mixture thereof. When used, the PPAR may make up from 0.001 to 2.5%, or from 0.003 to 1.8%, or from 0.004 to 1%, or from 0.005 to 00.8% by weight of the lamellar wash composition.

In another embodiment of the invention, the wash composition of the present invention comprises a skin prolipid rejuvenating mix comprising a C₁₄₋₁₆ fatty acid (preferably palmitic acid), glycerol, and PPAR such as 12-hydroxystearic acid, petroselinic acid, conjugated linoleic acid, stearic acid or a mixture thereof. When used, the weight percent of the skin prolipid rejuvenating mix, collectively, in the wash compositions is from 0.001 to 6%, or from 0.005 to 5%, or from 0.005 to 4.5%, or from 0.006 to 2.5% or from 0.007 to 1.5% or from 0.008 to 1% or from 0.008 to 0.8% or from 0.008 to 0.5% by weight of the cleansing composition.

In an often preferred embodiment of the invention, at least one of the following optional ingredients selected from the group consisting of carageenan, palmitic acid, 12-hydroxstearic acid, 10-hydroxystearic acid, lauric acid, glycolic acid, mandelic acid, caffeine, squalene, farnesol, neem oil, ceramide (N-acylsphingosine), niacinamide, retinyl propionate, Vitamin E (including tocopherol acetate, tocopheryl linoleate, tocopheryl oleate, tocopheryl nicotinate), Vitamin C (including sodium ascorbyl phosphate, ascorbyl glucoside, ascorbyl palmitate, ascorbyl tetraisopalmitate), 4-ethyl resorcinol, 4-hexyl resorcinol, salicylic acid, terpineol, thymol, thiamidol, benzalkonium chloride, benzethonium chloride, hyaluronic acid, hydrolyzed hyaluronic acid, collagen, pantethenol, climbazole, B1-thiamin, B-2 riboflavin, B-5 pantothenic acid, B6-pyroxidone, B7-biotin, B9, folic acid, pentavitin, magnolia bark extract, ginger extract, tetrahydroxypropyl ethylenediamine and a mixture thereof is used in the lamellar wash composition. If used, and again, those which are oil soluble are present at up to 2% by weight of the lamellar wash composition whereby in an embodiment of the invention those which are water-soluble when optionally used may make up to 10% by weight of the lamellar wash composition.

In yet another embodiment, at least one of soybean oil, hydrogenated soybean oil, sunflower seed oil, shea butter or a mixture thereof is used in the compositions of the invention.

In still another embodiment of the invention, the lamellar cleansing composition of the present invention can comprise at least one of S-adenosyl-L-methionine, a 1-alkyl nicotinamide having structure X, and a methionine having structure XI:

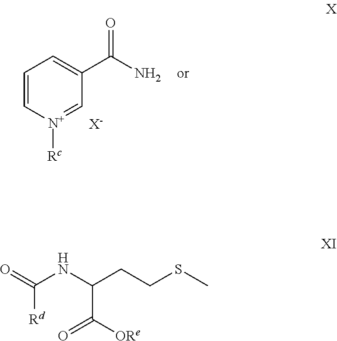

wherein R^{c} is a C₁₋₄ alkyl, preferably, methyl (N-methyl nicotinamide) and X⁻ is a negative counter ion, preferably, Cl—, R^{d} is methyl, ethyl, propyl, hydroxymethyl, 2-hydroxyethyl, preferably, methyl and R^{e} is H, methyl, ethyl, isopropyl, preferably, H (N-acetyl methionine). Such components are described in WO/2021/008824A1, the disclosure of which is incorporated herein by reference. When used, from 0.0001 to 10% or from 0.001 to 8%, and most preferably, from 0.01 to 5% or from 0.01 to 3% or from 0.01 to 2% or from 0.01 to 1% by weight of each of such ingredients are used. In even another embodiment, the lamellar cleansing composition may comprise acetyl cysteine at from 0.001 to 1.8% by weight of the cleansing composition.

In another embodiment of the invention and when making the lamellar cleansing composition of the present invention, the desired ingredients may be mixed with conventional apparatus under moderate shear and atmospheric conditions, with temperature ranging from 30 to 85° C. whereby shear continues until a homogeneous product is recovered. As previously noted, oil or (nanoemulsion) having a droplet size of less than 1 micron is preferably provided as an ingredient as an emulsion with submicron droplets being homogeneously dispersed in the lamellar wash.

When using the wash composition of the present invention, a surface, like skin, is contacted (in no particular order) with water and the lamellar cleansing composition. Shear is applied subsequently or simultaneously to generate a superior lather, and the resulting lathered composition is then wiped off, rinsed with water or both. Surprisingly, and when the surface being washed is skin, the consumer experiences a clean wash with skin not feeling tacky and enjoys excellent moisturizing sensory attributes. When consumers are washing, the ratio of cleaning composition to water is typically from 1:3 to 3:1, or from 1:2 to 2:1, or from 1:1.5 to 1.5:1, or from 1:1.2 to 1.2:1 or 1:1. The water used is often from about 5 to 55° C., or from 7 to 50° C., or from 12 to 50° C., or from 20 to 38° C.

The packaging for the lamellar cleansing composition typically is not limited as long as composition can be dispensed. In an embodiment of the invention, the wash composition is sold in a pouch, bottle (glass, metal or plastic), jar, tube, vessel with a pumpable actuator or canister. The packaging preferably is refillable, biodegradable and/or is prepared from recycled materials including post-consumer resins. Other packaging often desired for use includes bottles referred to as paper bottles that typically comprise at least 85% by weight paper. Even other packaging suitable for use includes bottles that comprise bamboo.

The Examples are provided to facilitate an understanding of the invention. They are not intended to limit the scope of the claims.

In the Examples, soybean oil not hydrogenated (i.e., not fully hydrogenated where the iodine value was 20 or less) had about 5 to 15% by weight of its carbon (hydrophobic) chain not fully saturated with hydrogen. Oil not provided in nanoemulsion (i.e., micro-oil) had a droplet size of about 10 microns.

### Example I

Nanoemulsions were made with the ingredients identified in Table I. The ingredients were combined and mixed with moderate shear, at atmospheric pressure, and while heating to about 58-75° C. to produce a macroemulsion. The resulting macroemulsion was sent via one pass through a high pressure homogenizer (nano DeBEE Laboratory Homogenizer, BEE International, MA, USA) with pressure at about 3,000 psi (20.7 MPa) to produce nanoemulsions (60% by weight total oil), Samples 1-8. In the nanoemulsions defined, water was added to balance. Hydrogenated Soybean oil (i.e., fully hydrogenated) had all of its carbon atoms saturated in its carbon (hydrophobic) chain. Where T-Cocamidopropyl betaine is identified, the same has 95-100% by weight of its hydrocarbon portion being triglyceride derived.

TABLE I

| Ingredients | Samples | | Sample | Samples | | Sample | Samples | |
|---|---|---|---|---|---|---|---|---|
| Nanoemulsion | 1 | 2 | 3 | 4 | 5 | 6 | 7 | 8 |
| Water | Balance | Balance | Balance | Balance | Balance | Balance | Balance | Balance |
| Soybean Oil | — | — | — | — | | 42 | — | — |
| Sunflower Seed Oil¹ | 60 | 60 | — | 48 | 48 | — | 36 | 36 |
| Hydrogenated Soybean Oil² | — | — | — | 12 | 12 | 18 | 24 | 24 |
| Shea Butter | — | — | 60 | — | — | — | — | — |
| Lauric Acid | 1.5 | 1.5 | 1.5 | 1.5 | 1.5 | 1.5 | 1.5 | 1.5 |
| Preservative | 0.5 | 0.5 | 0.5 | 0.5 | 0.5 | 0.5 | 0.5 | 0.5 |
| Na Lauroyl Isethionate | 5.4 | 5.4 | 5.4 | 5.4 | 5.4 | 5.4 | 5.4 | 5.4 |
| Na Methyl Lauroyl Taurate | — | 1.9 | — | 1.9 | — | 1.9 | — | 1.9 |
| T-Cocamidopropylbetaine³ | 2.1 | — | 2.1 | — | 2.1 | — | 2.1 | — |
| D[4,3] nm | 212 | 235 | 170 | 205 | 209 | 210 | 209 | UM-G |
| pH | 6.2 | 6.6 | 6.3 | 6.7 | 6.2 | 6.4 | 6.3 | 6.6 |
| Stability Pass | Yes | Yes | Yes | Yes | Yes | Yes | No | No |

¹high (80%) oleic acid, monosaturated;
²saturated, fully hydrogenated;
³hydrophobic portion triglyceride derived
UM-G—Unmeasured, gelled

The results in Table I surprisingly show that when nanoemulsions are made according to the invention, Samples 1 to 6, a stable nanoemulsion that remains pourable, free of precipitate and free of syneresis even after being stored for 12 months at 25° C. Further surprising was the fact that the nanoemulsions made according to the invention remained stable with surfactant levels that did not exceed 11.5% and the oil present was as high as 60% by weight of the nanoemulsion. The nanoemulsions had a viscosity from 10,000 to 20,000 mPa-s at 25° C. with shear rate of 4s-1.

Samples 7 and 8, not prepared consistent with the invention, displayed severe gellation, making the Samples unusable to include in a manufacturing process designed for making lamellar cleansing compositions. Such Samples failed, demonstrating that the ratio of oil to fully hydrogenated oil is essential to produce the superior nanoemulsion of the present invention.

### Example II

Lamellar wash compositions were made by mixing the ingredients identified in the Table II. All compositions had water added to balance. The pH of the lamellar wash compositions ranged from 6.2 to 6.9 and the viscosity was measured as described with a Discovery HR-2 Rheometer. When making the Lamellar wash compositions described in Table II, oil and water phases were made with mixing, achieved with moderate shear, at atmospheric pressure and heat supplied from 40 to 75° C. The resulting phases were also combined and mixed under such conditions and all ingredients were subsequently added. Mixing stopped when a homogeneous wash composition was produced. The numbers provided are based on total weight of the lamellar wash composition and ingredient as active. The starch used was sodium hydroxypropyl starch phosphate. Compositions made according to the invention were stored for 2 weeks at 50° C. After visual assessment, viscosity remained consistent and no precipitate, syneresis or flocculation was observed, confirming the compositions according to the invention were very stable. Oil added via a nanoemulsion (at about 40° C.) had a droplet size of about 200 nm where the nanoemulsion was incorporated as an ingredient when making the lamellar body wash. The weight percents in Table II associated with the nanoemulsions reflect the actual amount of nano-oil in the end use composition, provided from a nanoemulsion that was 60% by weight oil.

TABLE II

| Ingredients | Sample 1 | Sample 2 | Sample 3 | Sample 4 | Sample 5 | Sample 6 |
|---|---|---|---|---|---|---|
| Water | Balance | Balance | Balance | Balance | Balance | Balance |
| Sodium Gluconate | 0.05 | 0.05 | 0.05 | 0.05 | 0.05 | 0.05 |
| Starch | 4.5 | 4.5 | 4.5 | 4.5 | 4.5 | 4.5 |
| Caprylyl Glycol | 0.3 | 0.3 | 0.3 | 0.3 | 0.3 | 0.3 |
| Sodium Lauroyl Glycinate | 2.5 | 2.5 | 2.5 | 2.5 | 2.5 | 2.5 |
| Sodium Lauroyl Isethionate | 2.5 | 2.5 | 2.5 | 2.5 | 2.5 | 2.5 |
| NaOH | 0.2 | 0.2 | 0.2 | 0.2 | 0.2 | 0.2 |
| Lauric Acid | 2.6 | 2.6 | 2.6 | 2.6 | 2.6 | 2.6 |
| Fully Hydrogenated Soybean | — | — | — | — | — | 1.2 |
| Oil | | | | | | |
| 12HSA | 0.01 | 0.01 | 0.01 | 0.01 | 0.01 | 0.01 |
| Sunflower Seed Oil | 2 | — | — | — | — | 0.8 |
| Stearic acid | 0.3 | 0.3 | 0.3 | 0.3 | 0.3 | 0.3 |
| Glycerin | 0.9 | 0.9 | 0.9 | 0.9 | 0.9 | 0.9 |
| Guar | 0.01 | 0.01 | 0.01 | 0.01 | 0.01 | 0.01 |
| T-Cocamidopropylbetaine | 5.2 | 5.2 | 5.2 | 5.2 | 5.2 | 5.2 |
| SFO Nanoemulsion¹ | — | 2 | — | — | — | — |
| SFO and FHO Nanoemulsion² | — | — | 2 | — | 5 | 3 |
| Shea Butter Nanoemulsion | — | — | — | 2 | — | — |
| NaCl | 0.3 | 0.3 | 0.3 | 0.3 | 0.3 | 0.3 |
| Sodium Benzoate | 0.5 | 0.5 | 0.5 | 0.5 | 0.5 | 0.5 |
| Citric Acid | 0.2 | 0.2 | 0.2 | 0.2 | 0.2 | 0.2 |
| Fragrance | 1 | 1 | 1 | 1 | 1 | 1 |
| Stability Pass³ | No | Yes | Yes | Yes | Yes | Yes |

¹SFO, sunflower seed oil;
²Sunflower Seed Oil (SFO) and fully hydrogenated soybean oil, 80% by weight of the oil mixture in the nanoemulsion being sunflower seed oil;
³Stability tested after storage for 2 weeks at 50° C.

As can be seen from the data in Table II, a lamellar cleansing composition (end use wash composition) not made consistent with the invention, e.g., Sample 1, was not stable and not suitable for use. The composition separated, displayed visual syneresis, as well as particulate or flocculate sedimentation, a direct result of oil not being added consistent with the invention.

Compositions made according to the present invention, were assessed and used by trained panelists. All concluded that the compositions made consistent with the invention, were homogeneous, displayed no syneresis and were creamy, consistent with the feel of a traditional skin lotion. Notwithstanding the lotion-like feel, all panelists unexpectedly concluded the compositions made according to the present invention lathered and rinsed off in a manner consistent with wash compositions having a total of less than 1% by weight oil. Furthermore, the panelist surprisingly noted that upon opening the package, the lamellar cleansing composition delivered strong and enduring fragrance notes in comparison to conventional wash compositions not made consistent with the invention.

Viscosity of the lamellar cleansing compositions was taken at 24 hours, 25° C. and at a shear rate of 0.1s-1. Sample 1 was not stable and viscosity, due to syneresis, was not measurable. For all other Samples the viscosity was as follows: Sample 2, 255,370 mPa-s; Sample 3, 520,200 mPa-s; Sample 4, 130,000 mPa-s; Sample 5, 1,630,000 mPa-s; Sample 6, 1,010,000 mPa-s.

What is claimed is:

1. An oil-in-water nanoemulsion comprising:
a) from 1.5 to 8.5% by weight anionic surfactant, the anionic surfactant comprising 100% by weight acyl isethionate or 100% by weight acyl taurate based on total weight of the anionic surfactant, or acyl isethionate, acyl taurate or both and co-anionic surfactant, the co-anionic surfactant making up from 0.01 to 65% by weight of the total weight of anionic surfactant in the nanoemulsion, the co-anionic surfactant not being an acyl isethionate or acyl taurate;
b) from 0.0 to 5.5% by weight amphoteric surfactant;
c) from 0.2 to 5.5% of a C₈-C₁₈, and preferably, C₁₀-C₁₆, and most preferably, C₁₂-C₁₄ fatty acid, fatty alcohol, fatty amide, or a mixture thereof;
d) from 40 to 70% by weight of a mixture of non-hydrogenated oil and hydrogenated oil, the mixture comprising no more than 37% by weight hydrogenated oil based on total weight of the mixture of non-hydrogenated and hydrogenated oil;
e) from 13 to 55% by weight water,
with the provisos that the nanoemulsion:
i) has a pH from 5.3 to 7.5, and preferably, from 6 to 7, and most preferably, from 6.1 to 6.9, or from 6.1 to 6.8;
ii) is substantially free of at least one of sulfate-based surfactants, phthalates, dioxanes, parabens, hydantoins and isothiazolinones;
iii) has total surfactant that does not exceed 11.5% by weight of the nanoemulsion;
iv) has the oil at a droplet size from 10 to 775 nm;
v) comprises from 42 to 75% by weight oil.

2. The nanoemulsion according to claim 1 wherein the anionic surfactant comprises only acyl isethionate, only acyl taurate or a mixture thereof, and further comprises at least one of soybean oil, hydrogenated soybean oil, fatty acid, shea butter and betaine.

3. A lamellar cleansing composition comprising:
a) anionic surfactant comprising from 1.8 to 6.75% by weight of acyl isethionate, and from 1.8 to 6.75% by weight of a co-anionic surfactant;
b) from 3 to 7.5% by weight amphoteric surfactant;
c) less than 1.25 by weight electrolyte;
d) from 0.3 to 6.5% by weight thickener based on total weight of the cleansing composition,
e) 0 to 4.5%, or from 0.001 to 4%, or from 0.01 to 3.5%, or from 0.1 to 3.3%, or from 1 to 3% by weight micro-oil;
f) nano-oil
with the provisos that:
i) at least 40% by weight total oil based on total weight of nano-oil and micro-oil in the cleansing composition is provided from nanoemulsion according to claim 1;
ii) the total weight of surfactant in the cleansing composition is from 6.5% to 12.3 by weight of the cleansing composition;
iii) the cleansing composition is substantially free of sulfate-based surfactant;
iv) at least 50% by weight of hydrophobic chain present on the amphoteric surfactant is triglyceride derived based on total weight of the hydrophobic chain on the amphoteric surfactant;
v) the cleansing composition has a pH from 5.0 to 7.5; and
vi) the cleansing composition comprises from 0.5 to 5.5% of a C₈-C₁₈ fatty acid, fatty alcohol, fatty amide, or a mixture thereof.

4. The lamellar cleansing composition according to claim 3 wherein the composition further comprises 12-hydroxystearic acid, 10-hydroxystearic acid, 2-hydroxystearic acid, petroselinic acid, conjugated linoleic acid or a mixture thereof.

5. The lamellar cleansing composition according to claim 3 wherein the anionic surfactant comprises a glycinate and isethionate.

6. The lamellar cleansing composition according to claim 3 wherein the wash composition comprises 8.5% by weight total oil or less based on total weight of the cleansing composition.

7. The lamellar cleansing composition according to claim 3 wherein the wash composition is substantially free of a sulfate-based surfactant and optionally comprises mandelic acid, caffeine, thiamidol or a mixture thereof.

8. The lamellar cleansing composition according to claim 3 wherein the wash composition comprises less than 35 ppm 1,4-dioxane and less than 1.25% by weight electrolyte.

9. The lamellar cleansing composition according to claim 3 wherein the composition further comprises a preservative, glycerol, sunscreen or a mixture thereof.

10. The lamellar cleansing composition according to claim 3 wherein the oil is not derived from petroleum.
